SEQUENCE LISTING (8 sequences)

SEQ ID NO: 1 - protein, 37 aa, Homo sapiens
  MOD_RES 28: note = phosphothreonine
AEPRQEFEVMEDHAGTYGLGDTPPAPKTPPSSGEPPK

SEQ ID NO: 2 - protein, 34 aa, Homo sapiens
DAGYEVHHQKLVFFAEDVGSEYPYDVPDYAAKLE

SEQ ID NO: 3 - protein, 32 aa, Homo sapiens
DYKDHDIDYKDDDDKGGGEYPYDVPDYAAKLE

SEQ ID NO: 4 - protein, 38 aa, Homo sapiens
  MOD_RES 30: note = phosphothreonine
AEPRQEFEVMEDHAGTYGLGDRSRTPSLPPTPPTREPK

SEQ ID NO: 5 - protein, 40 aa, Homo sapiens
DAEFRHDSGYEVHHQKLVFFAEDVGSNKGAIIGLMVGGVV

SEQ ID NO: 6 - protein, 42 aa, Homo sapiens [sequence omitted]

SEQ ID NO: 7 - protein, 37 aa, Homo sapiens; identical to SEQ ID NO: 1

SEQ ID NO: 8 - protein, 37 aa, Homo sapiens
AEPRQEFEVMEDHAGTYGLGDRSRTPSLPTPPTREPK

(19) United States
(12) Patent Application Publication
(10) Pub. No.: US 2026/0029414 A1
(43) Pub. Date: Jan. 29, 2026

(54) HIGH PRECISION AND COST-EFFECTIVE MULTIPLEX QUANTIFICATION OF AB40, AB42, P181TAU, P217TAU, NFL, AND GFAP FROM PLASMA AND SERUM

(71) Applicant: University of Southern California, Los Angeles, CA (US)

(72) Inventors: Ebrahim ZANDI, Los Angeles, CA (US); Farshad ALISHAHI, Los Angeles, CA (US); Christopher Ryan BEAM, Los Angeles, CA (US)

(73) Assignee: University of Southern California, Los Angeles, CA (US)

(21) Appl. No.: 19/222,259

(22) Filed: May 29, 2025

Related U.S. Application Data
(60) Provisional application No. 63/676,519, filed on Jul. 29, 2024.
(60) Provisional application No. 63/754,717, filed on Feb. 6, 2025.

(51) Int. Cl. G01N 33/68; G01N 33/543

(52) U.S. Cl. CPC G01N 33/6896; G01N 33/54386; G01N 2800/2821

(57) ABSTRACT

A bioassay system for multiplexed detection and quantification of multiple analytes (e.g., Aβ40, Aβ42, pTau181, p217Tau, GFAP, and NFL) in a biological sample is provided. The bioassay system includes a plurality of sets of color-coded microspheres. Each set of microspheres is distinguishable by a unique color code generated by internal dyes. The bioassay system includes a first set of control microspheres attached to mouse polyclonal IgG to correct for a background of individual specimens and a second set of control microspheres configured to capture a synthetic peptide to normalize for well-to-well variations. Bioassay system also includes a fluidic system configured to mix the sample with the plurality of sets of color-coded microspheres to allow for specific binding between analytes and their corresponding capture agent among other analytes and a detection system for exciting and reading fluorescence of the internal dyes and a reporter fluorescence indicative of analyte binding.

### CROSS-REFERENCE TO RELATED APPLICATIONS

This application claims the benefit of U.S. provisional application Ser. No. 63/676,519 filed Jul. 29, 2024, and the benefit of U.S. provisional application Ser. No. 63/754,717 filed Feb. 6, 2025, the disclosures of which are hereby incorporated in their entirety by reference herein.

### STATEMENT REGARDING FEDERALLY SPONSORED RESEARCH OR DEVELOPMENT

This invention was made with government support under Grant Nos. AG066530, AG054442, AG060049, and AG063949 awarded by the National Institutes of Health (NIH). The government has certain rights in the invention.

### SEQUENCE LISTING

The text file Sequence_USC0377PUSP.xml of size 8,755 bytes created Oct. 10, 2025, filed herewith, is hereby incorporated by reference.

### TECHNICAL FIELD

In at least one aspect, the present invention is related to methods and systems for detecting biomarkers, and in particular, Alzheimer's biomarkers.

### BACKGROUND

There are several methods for quantification of plasma biomarkers including Aβ40, Aβ42, and phosphorylated tau (mainly p181Tau and p217Tau), neurofilament light chain (NfL), and glial fibrillary acidic protein (GFAP) for AD and other neurological disorders¹. Plasma Aβ42/Aβ40 and p217Tau, p181Tau/Aβ42 ratios, NfL, and GFAP correlate well with amyloid PET or CSF and plasma values, and some may predict clinical disease progression²⁻⁸. The p217Tau has been shown to have the highest diagnostic accuracy for AD among other biomarkers^{6-7,9-10}. The methods vary in terms of availability, sensitivity, multiplexing capability, cost, equipment, requirement of high levels of technical expertise, and high-throughput capability (reviewed in¹). In general, the high cost and wide availability of these assays maybe a barrier for large-scale studies needed for ultimate validation of blood-based biomarkers for diagnosis and prognosis of AD not only in industrialized and wealthy countries but worldwide^{1,11}.

Luminex xMAP-based technology is utilized widely for multiplex quantification of protein biomarkers from plasma and serum. Luminex xMAP has been used to quantify Aβ40, Aβ42 and p181Tau in CSF¹² and plasma^{13,14}. Park et al employed Luminex xMAP to quantify Aβ40 and Aβ42 from protease and phosphatase treated plasma and showed lower Aβ42/Aβ40 plasma ratios are associated with positive cerebral amyloid depositions and AD diagnosis compared to subjects with negative cerebral amyloid depositions and non-AD diagnosis¹³. Lovheim et al utilized Luminex xMAP technology to quantify Aβ42 and Aβ40 from plasma of several hundred preclinical AD cases and control subjects¹⁴.

### SUMMARY

In another aspect, a bioassay system for multiplexed detection and quantification of multiple analytes in a biological sample in a refinement, the bioassay system includes a plurality of sets of color-coded microspheres. The plurality of sets of color-coded microspheres includes a first set of microspheres distinguishable by a first unique color code generated by internal dyes and is coated with a specific capture agent that binds to Aβ40, a second set of microspheres distinguishable by a second unique color code generated by internal dyes and is coated with a specific capture agent that binds to Aβ42, a third set of microspheres distinguishable by a third unique color code generated by internal dyes and is coated with a specific capture agent that binds to pTau181 or p217Tau, a fourth set of microspheres distinguishable by a fourth unique color code generated by internal dyes and is coated with a specific capture agent that binds to GFAP, and a fifth set of microspheres distinguishable by a fifth unique color code generated by internal dyes and is coated with a specific capture agent that binds to Neurofilament Light Chain (NFL). The system can also include a fluidic system configured to mix the biological sample with the plurality of sets of color-coded microspheres to allow for specific binding between analytes and their corresponding capture agent among other analytes and their respective capture agents and to introduce reporter molecules that bind to captured analytes. Characteristically, the reporter molecules configured to emit a detectable reporter fluorescence upon excitation. The system can also include a detection system including a flow cell and capable of exciting and reading fluorescence of each internal dye and the detectable reporter fluorescence indicative of analyte binding. Advantageously, the detection system is configured to identify each of the sets of microspheres by its associated unique color code and quantify bound analytes based on the detectable reporter fluorescence, alongside quantification of other analytes.

In another aspect, the third unique color code generated by internal dyes and is coated with a specific capture agent that binds to p217Tau.

In another aspect, the specific capture agents for Aβ40, Aβ42, either pTau181 or p217Tau, GFAP, and NFL are antibodies that specifically bind to their respective analytes, allowing for selective detection and quantification of these analytes in the presence of other analytes within the biological sample.

In another aspect, the development of a Penta-Plex Alzheimer's Disease Capture Sandwich Immunoassay (5ADCSI) based on Luminex xMAP technology and commercially available antibodies for multiplex quantification of Aβ40, Aβ42, p181Tau, p217Tau, NfL, and GFAP in CSF, plasma and serum is provided. To better correct for the background signal and to reduce well-to-well variability in each well, we designed and included MagPlex microspheres attached to mouse polyclonal IgG (mIgG) and microspheres to capture a synthetic peptide (HJ-HA, see below), added to each sample. The specificity of each capture/detection antibody pair is verified in this assay by examining specificity/cross reactivity of each analyte for their corresponding antibodies, and specific immune depletion of human plasma. We validated the utility of 5ADCSI in matched CSF and plasma or serum for differentiating cognitively normal (CN) from those with mild cognitive impairment (MCI) and demented due to Alzheimer's disease (AD).

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIGS. 1A and 1B. Schematics of a bioassay system for detecting and quantifying multiple analytes in a biological sample.

FIGS. 2A, 2B, and 2C. List of peptides used as a control.

FIG. 3. Schematic of microbeads used in the system of FIGS. 1A and 1B.

FIGS. 4 Aa, 4Ab, 4Ac, 4Ad, 4Ba, 4Bb, 4C, 4Da, 4Db, 4Dc, 4Dd, 4De, and 4Df. Specificity of antibodies for detection of Aβ40, Aβ42, p181Tau, p217Tau, NfL, and GFAP peptides in human plasma. A, Antibodies used for the 5ADCSI method are specific for their corresponding target peptides. Specificity of each antibody was examined in 20 mg/ml BSA in combinations of indicated peptides (range of 10-10000 μg/ml). Data are presented as mean±SD of triplicate measurements. At the top of each plot the antibody coupled to the MagPlex microspheres is indicated. B, Schematic presentation of unphosphorylated and p181, p217 phosphorylated tau peptides, respectively, used in A to examine the specificity of p181Tau and p217Tau antibodies. Tau12 is a N-terminal peptide of human tau protein used as the epitope for detection antibody in the assay. C, Phosphatase treatment of brain extract of a subject with AD indicates specificity of p181Tau and p217Tau for detection of endogenous phospho-Tau species. D, specific immune depletion of each analyte in human plasma indicates specificity of each antibody for their corresponding analyte in human plasma. See Result section for details of the experiment. Below of each bar graph the antibodies used for immune depletion are indicated. Aβ40: Amyloid beat 40, Aβ42: Amyloid beta 42, p181Tau: phosphorylated tau at position threonine 181, p217Tau: phosphorylated tau at position threonine 217, NfL: Neurofilament light chain, GFAP: glial fibrillary acidic protein, IgG, mouse immune globulin (control antibody), MFI, median fluorescence intensity.

FIGS. 5A, 5B, 5C, 5D, 5E, and 5F. Detection sensitivity of Aβ40, Aβ42, p181Tau, p217Tau, NfL, and GFAP in plasma. Data illustrate the sensitivity of detection of Aβ40, Aβ42, p181Tau, p217Tau, NfL, GFAP spiked in range of (0-8 μg/ml analyte per 20 mg/ml plasma for Aβ40, Aβ42, and GFAP, and 0-2 fmol/ml per 20 mg/ml plasma for p181Tau, p217Tau, and 0-1.3 fmol/ml per 20 mg/ml plasma for NfL) in five different human plasmas. The data indicates the sensitivity and consistency of the 5ADCSI in detecting each analyte above various endogenous amounts of each biomarker.

FIGS. 6A, 6B, and 6C. Biomarker analysis of matching cerebral spinal fluid (CSF) and plasma or serum samples from individuals diagnosed with Alzheimer's disease (AD), mild cognitive impairment (MCI), and cognitively normal (CN). Each panel from left to right shows the name of the biomarker, a bar graph of the mean quantified biomarker values, the receiver-operating characteristic curve (ROC) with the corresponding area under the curves (AUC) values for CSF and plasma or serum. ROCs show MCI versus CN, and AD versus CN. Aβ42/Aβ40 ratio, p181Tau: phosphorylated tau at position threonine 181, p217Tau: phosphorylated tau at position threonine 217, p217Tau/Aβ42 ratio, p181Tau/Aβ42 ratio, and GFAP: glial fibrillary acidic protein. * P<0.05, **P<0.001, ***P<0.0001, ****P<0.00001

FIGS. 7A, 7B, 7C, 7D, 7E, and 7F. Pearson's correlations of AD biomarkers between CSF and plasma/serum. Pair-wise Pearson's correlation coefficient (R) and corresponding p-values of the ratios of Aβ42/40, p217Tau, p181Tau, p217Tau/Aβ42, p181Tau/Aβ42, and GFAP between CSF and serum/plasma are shown. The y-axis indicates the mean CSF values, and the x-axis indicates the mean plasma values. The green circles indicate subjects diagnosed as cognitively normal (CN). The red circles indicate subjects diagnosed with dementia due to Alzheimer's disease (AD). The blue circles indicate subjects diagnosed with Mild cognitive impairment (MCI). The correlation coefficient (R) and p-values are shown in the upper left corner of each graph. The p-values smaller than 0.05 show significant correlation of CSF and plasma or serum measurements. Abbreviations: CSF: cerebrospinal fluid; Aβ: amyloid β; p217Tau: phosphorylated tau at position threonine 217; p181Tau: phosphorylated tau at position threonine 181; GFAP: glial fibrillary acidic protein.

FIG. 8. Boxplots of SURV Measures by Amyloid status. Boxplots depict diagrams of standardized uptake value ratios (SUVRs) for ¹⁸F-florbetaben (FBB) PET scans shown Table 2. SUVR values are plotted versus amyloid status.

FIGS. 9A, 9B, 9C, and 9D. Biomarker analysis of matching cerebral spinal fluid (CSF) and plasma or serum samples from individuals with amyloid positive (Ab+) and negative (Ab−). Each panel from left to right shows the name of the biomarker, a bar graph of the mean quantified biomarker values, the receiver-operating characteristic curve (ROC) with the corresponding area under the curves (AUC) values for CSF and plasma or serum. ROCs show Ap-versus Aβ+ status. Aβ40: Amyloid beta 40, Aβ42: Amyloid beta 42, Aβ42/Aβ40 ratio, p181Tau: phosphorylated tau at position threonine 181, p217Tau: phosphorylated tau at position threonine 217, p217Tau/Aβ42 ratio, p181Tau/Aβ42 ratio, GFAP: glial fibrillary acidic protein, NfL: Neurofilament light chain. * P<0.05, **P<0.001, ***P<0.0001, ****P<0.00001

FIGS. 10A, 10B, 10C, 10D, 10E, 10F, and 10G. Pearson's correlations of AD biomarkers between CSF and plasma/serum. Pair-wise Pearson's correlation coefficient (R) and corresponding p-values of the of Aβ42, Aβ40, Aβ42/40, p217Tau, p181Tau, and GFAP between CSF and serum/plasma are shown. The y-axis indicates the mean CSF values, and the x-axis indicates the mean plasma values. The color rad indicates PET amyloid negative, and green PET amyloid positive. The square shape indicates subjects diagnosed as cognitively normal (CN). The square shape indicates subjects diagnosed with dementia due to Alzheimer's disease (AD). The triangle shape indicates subjects diagnosed with Mild cognitive impairment (MCI). The correlation coefficient (R) and p-values are shown in the upper left corner of each graph. The p-values smaller than 0.05 show significant correlation of CSF and plasma or serum measurements. Abbreviations: CSF: cerebrospinal fluid; Aβ: amyloid β; p217Tau: phosphorylated tau at position threonine 217; p181Tau: phosphorylated tau at position threonine 181; GFAP: glial fibrillary acidic protein.

FIG. 11. Table 1: Demographic and biomarker data of the cohort with matched CSF, plasma, and serum.

FIG. 12. Table 2: PET amyloid status and etiological diagnosis based on CDR (The Clinical Dementia Rating) and MoCA (The Montreal Cognitive Assessment) scores for 16 subjects.

FIG. 13. Table 3: Biomarker data fof the subjects with PET-amyloid status as stratification criterion.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e., the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only B, or both A and B.” In the case of “only A,” the term also covers the possibility that B is absent, i.e. “only A, but not B.”

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The terms “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entirety are hereby incorporated by reference into this application to more fully describe the state of the art to which this invention pertains.

To determine the “percent identity” (i.e., percent sequence identity) of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a refinement, the sequences are aligned for maximum correspondence over a specified comparison window, as measured by sequence comparison algorithms or by visual inspection. In a refinement, the length of a first sequence aligned for comparison purposes is at least 80% of the length of a second sequence and, in some embodiments, is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extends penalty of 4, and a frameshift gap penalty of 5. In this regard, the following oligonucleotide alignment algorithms may be used: BLAST (GenBank URL: www.ncbi.nlm.nih.gov/cgi-bin/BLAST/, using default parameters: Program: BLASTN; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSUM62; Filter: default, echofilter: on, Expect:10, cutoff: default; Strand: both; Descriptions: 50, Alignments: 50), or FASTA, search, using default parameters. When sequences differ in conservative substitutions, the percent identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. In any peptide sequence provided herein, sequences with at least 70%, 80%, 90%, 95%, 98%, or 99% identity can be alternatively used. The peptides provided herein are synthetic.
“Aβ” means amyloid beta.
“Aβ40” means amyloid beta 40.
“Aβ42” means amyloid beta 42.
“ACD” means all-cause dementia.
“AD” means Alzheimer's disease.
“ADRC” means Alzheimer's Disease Research Center.
“ALS” means amyotrophic lateral sclerosis.
“ANTs” means Advanced Normalization Tools.
“AUC” means area under the curve.
“BSA” means bovine serum albumin.
“CDR” means Clinical Dementia Rating.
“CDR-SB” means Clinical Dementia Rating-Sum of Boxes.
“CHAPS” means 3-[(3-Cholamidopropyl) dimethylammonio]-1-propanesulfonate
hydrate.
“Cr” means confidence interval.
“CN” means cognitively normal.
“CSF” means cerebrospinal fluid.
“EDC” means 1-ethyl-3-[3-dimethylaminopropyl]carbodiimide hydrochloride.
“EDTA” means ethylenedinitrilo)tetraacetic acid.
“EPPS” means 4-(2-Hydroxyethyl)piperazine-1-propanesulfonic acid.
“FBB” means 18F-florbetaben.
“GFAP” means glial fibrillary acidic protein.
“HA” means hemagglutinin.
“HJ-HA” means hybrid peptide composed of HJ5.1 and HA epitopes.
“IC45” means MagPlex® Monitoring Microspheres for instrument calibration.
“IgG” means immunoglobulin G.
“IIR” means Immunoglobulin Inhibiting Reagent.
“IRB” means Institutional Review Board.
“LoB” means limit of blank.
“LoD” means limit of detection.
“LoQ” means limit of quantification.
“mIgG” means mouse immunoglobulin G.
“MCI” means mild cognitive impairment.
“MES” means 2-(N-morpholino)ethanesulfonic acid.
“MFI” means median fluorescence intensity.
“MoCA” means Montreal Cognitive Assessment.
“MRI” means magnetic resonance imaging.
“MS” means multiple sclerosis.
“NFL” means neurofilament light chain.
“NHS” means N-hydroxysulfosuccinimide.
“PBS” means phosphate-buffered saline.
“PET” means positron emission tomography.
“ROI” means region of interest.
“SAPE” means Streptavidin R-phycoerythrin conjugate.
“SD” means standard deviation.
“SUVR” means standardized uptake value ratio.
“TBI” means traumatic brain injury.
“VaD” means vascular dementia.
“xMAP” means Multi-Analyte Profiling.

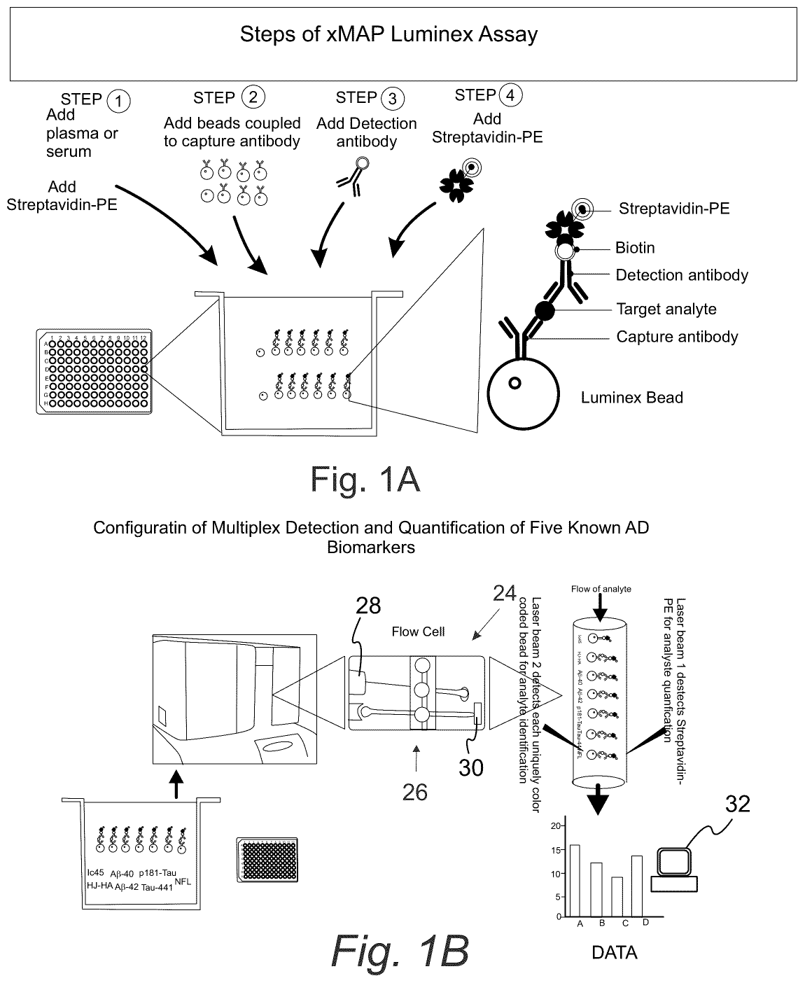

With reference to FIGS. 1A and 1B, a bioassay system for multiplexed detection and quantification of multiple analytes in a biological sample is provided. In a first embodiment, a bioassay system 10 for multiplexed detection and quantification of multiple analytes in biological samples is provided. The system utilizes a bead-based technology featuring sets of color-coded microspheres 12, each distinguishable by unique internal fluorescent dye combinations that create specific spectral signatures. As shown in FIG. 1A, the system can follow a sandwich immunoassay protocol where each microsphere set is coated with a specific capture agents that selectively bind to predetermined target analytes. The biological sample, which can be cerebrospinal fluid sample, a plasma sample, or a serum sample, is mixed with these functionalized microspheres in a multi-well plate format. FIG. 1B illustrates how the system processes these microsphere-analyte complexes through a flow cell 26 where dual lasers 28, 30 simultaneously identify the bead type via the color code and quantify the amount of bound analyte through reporter fluorescence intensity. This multiplex system incorporates control microspheres to normalize signals, correct for background, and ensure reproducibility across measurements, enabling simultaneous analysis of multiple biomarkers from a single sample.

In another aspect, the plurality of sets of color-coded microspheres includes a first set of microspheres that is distinguishable by a unique color code generated by internal dyes and is coated with a specific capture agent that binds to Aβ40, a second set of microspheres that is distinguishable by a unique color code generated by internal dyes and is coated with a specific capture agent that binds to Aβ42, and a third set of microspheres that is distinguishable by a unique color code generated by internal dyes and is coated with a specific capture agent that binds to pTau181 or p217Tau. In a refinement, the plurality of sets of color-coded microspheres also includes a fourth set distinguishable by a fourth unique color code and coated with a specific capture agent that binds to GFAP. In a further refinement, the plurality of sets of color-coded microspheres also includes a fifth set distinguishable by a fifth unique color code and coated with a specific capture agent that binds to Neurofilament Light Chain (NFL). Bioassay system 10 also includes a fluidic system 22 configured to mix the sample with the plurality of sets of color-coded microspheres to allow for specific binding between analytes and their corresponding capture agent among other analytes and their respective capture agents. Bioassay system 10 also includes a detection system 24 that includes a flow cell 26 and is capable of exciting and reading fluorescence of the internal dyes and a reporter fluorescence indicative of analyte binding. In particular, the detection system can includes a first laser 28 for exciting internal dyes and a second laser 30 for exciting reporter fluorescence. Advantageously, the detection system is configured to identify each of the sets of microspheres by the unique color code and quantify bound analytes based on the reporter fluorescence, alongside quantification of other analytes.

In another aspect, the third set of microspheres is distinguishable by a unique color code generated by internal dyes and is coated with a specific capture agent that binds to p217Tau. Advantageously, p217Tau is a highly accurate biomarker for diagnosing Alzheimer's disease (AD), outperforming p181Tau in distinguishing mild cognitive impairment (MCI) and AD from cognitively normal (CN) individuals. As shown below, using the newly developed 5ADCSI multiplex assay, p217Tau in cerebrospinal fluid (CSF), plasma, and serum can be quantified, showing strong correlation between CSF and blood levels. p217Tau levels were significantly elevated in MCI and AD patients, with an AUC of 0.95 in plasma/serum, indicating high diagnostic accuracy. The p217Tau/Aβ42 ratio further improved classification, achieving an AUC of 0.94 in plasma/serum and 0.99 in CSF. Additionally, p217Tau levels correlated with amyloid PET positivity, with amyloid-positive individuals showing 2.48-fold (CSF) and 4.7-fold (plasma/serum) higher p217Tau levels. These findings support p217Tau as a superior blood-based biomarker for AD, enabling cost-effective, large-scale screening and early diagnosis.

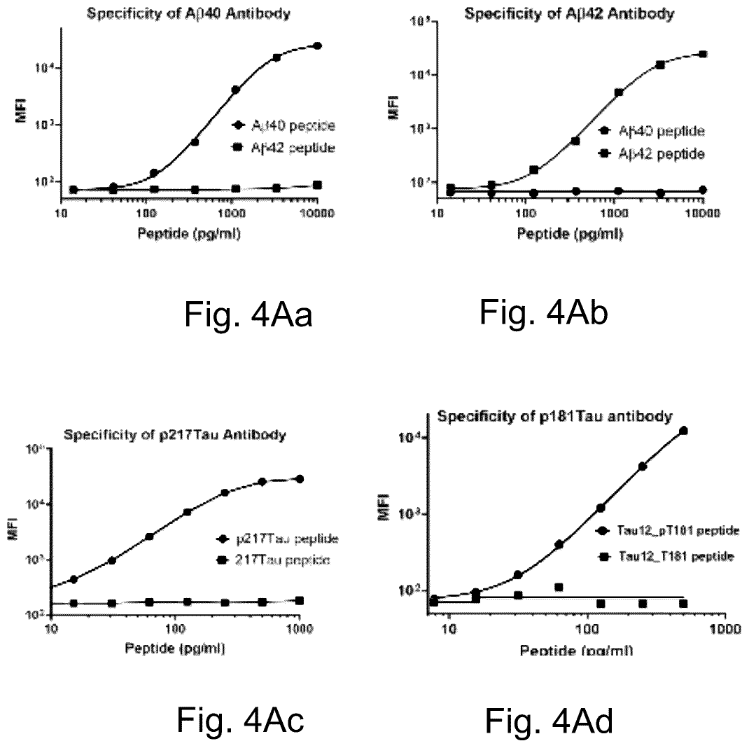

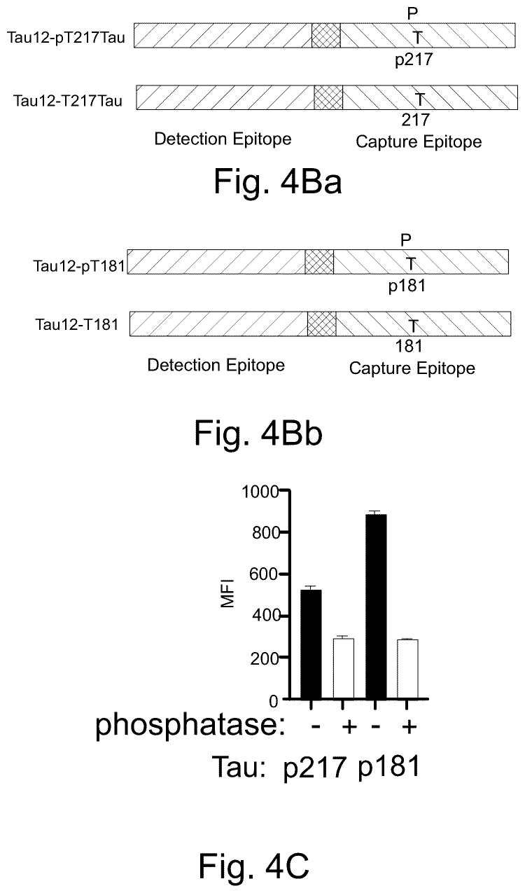

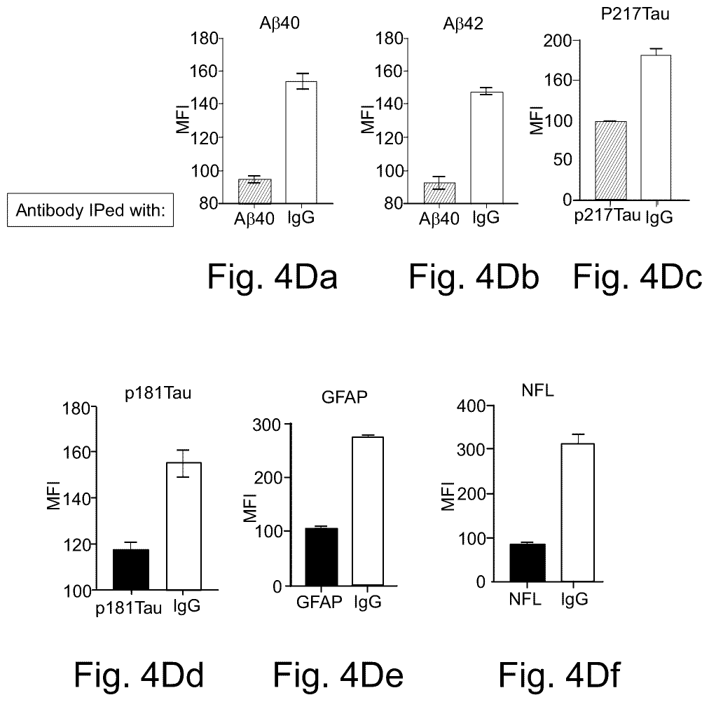

In another aspect, a peptide for specificity testing and as a calibrator for p217Tau having sequence AEPRQEFEVMEDHAGTYGLGDRSRTPSLP(pT)PPTREPK (SEQ ID NO: 4) or a peptide at least 70%, 80%, 85%, 90%, 95%, 97%, or 99% identical to SEQ ID NO: 4. This synthetic peptide comprises two key epitopes: the N-terminal epitope recognized by the detection antibody (Tau12) and the phosphorylated threonine at position 217 recognized by the capture antibody (E9Y4S). The peptide enables accurate calibration of the assay by providing a precisely phosphorylated standard with consistent solubility, overcoming limitations associated with recombinant tau proteins that suffer from incomplete phosphorylation and solubility issues. As demonstrated in FIG. 4A, antibodies used in the 5ADCSI method show high specificity for their corresponding target peptides, with the p217Tau capture microspheres selectively identifying the phosphorylated Tau12-pTau217 peptide but not the non-phosphorylated Tau12-T217 peptide. This specificity was further confirmed through phosphatase treatment experiments and immune depletion tests, ensuring reliable detection of endogenous p217Tau in clinical samples.

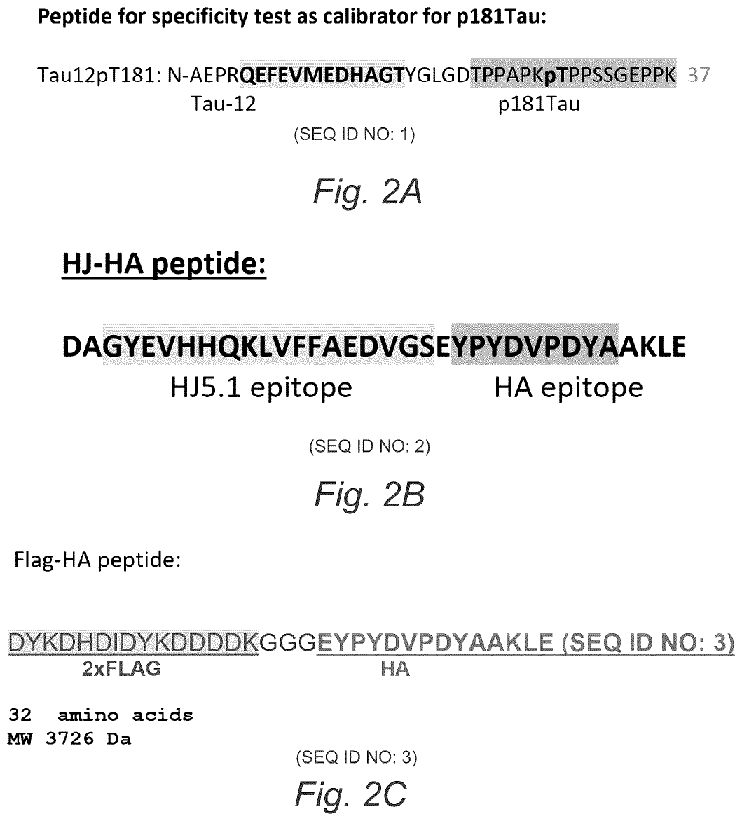

In another aspect, a peptide for specificity testing and as a calibrator for p181Tau having sequence N-AEPRQEFEVMEDHAGTYGLGDTPPAPKpTPPSSGEPPK (SEQ ID NO: 1) or a peptide having at least 90% or 95% identical to SEQ ID NO: 1. FIG. 2B provides peptide SEQ ID: 1 with the Tau-12 epitope and the P181Tau epitope highlighted. The Tau-12 epitope, recognized by the N-terminal mouse monoclonal antibody Tau12, is within the first 22 amino acids of human Tau (Uniport: P10636-8, 2N4R protein). The exact amino acid sequence is QEFEVMEDHAGT. The antibody is purchased from Biolegend. The P181Tau epitope encompasses amino acids 175 to 191 of human Tau (Uniport: P10636-8, 2N4R protein) with the phosphorylated form of threonine 181 residue. We use Phospho-Tau (Thr181) Monoclonal Antibody (AT270) from Thermo Fischer for our assay. The control peptide Tau12T181 is exactly the same as the Tau12pT181 with the exception that threonine 181 is not phosphorylated. In a refinement, the sequences with at least 90% or 95% identity or any specified identity will not have any substitutions in either epitope. In a further refinement, there are 1, 2, or 3 conservative substitutions in non-epitope positions.

In another aspect, the synthetic peptide includes a peptide having sequence DAGYEVHHQKLVFFAEDVGSEYPYDVPDYAAKLE (SEQ ID NO: 2) referred to as HJ-HA peptide or a peptide at least 90% or 95% identical to SEQ ID NO: 2. Referring to FIG. 2A, the HJ-HA is a combination of two epitopes. The N-terminal sequence (HJ5.1 epitope) encompasses amino acids 9-28 of human amyloid beta proteins. The hybridoma cell lines for producing antibody HJ5.1 was obtained from Dr. David Holtzman. This is the same capture antibody we also use for quantification of Aβ40 and Aβ42 in our assay. One reason to use this antibody in the control peptide (HJ-HA) was to capture and account for potential backgrounds of Aβ40 and Aβ42 analytes. The Hemagglutinin (HA) is a common epitope (highlighted in green) used to tag protein when expressing recombinantly because the antibodies are commercially available and widely used. The amino acid sequences not highlighted are added as random sequences. In a refinement, the sequences with at least 90%, 95%, or 99% identity or any specified identity will not have any substitutions in either epitope. In a refinement, there are 1, 2, or 3 conservative substitutions in non-epitope positions. Peptides Aβ40 and Aβ42 were purchased from Anaspec and sequences are exactly as they occur in human.

In another aspect, the synthetic peptide includes a peptide having sequence DYKDHDIDYKDDDDKGGGEYPYDVPDYAAKLE (SEQ ID NO: 3) referred to as Flag-HA peptide. The peptide has 32 amino acids and a molecular weight of 3726 Daltons. (Description of FLAG tag: https://resources.chromotek.com/blog/flag-tag-and-3x-flag-tag-an-epitope-tag-for-capture-and-detection-experiments.) The FLAG-HA is a combination of two epitopes. The synthetic FLAG epitope is highlighted in FIG. 2C. Antibodies to this epitope are commercially available. The Hemagglutinin (HA) is a common epitope used to tag protein when expressing recombinantly because the antibodies are commercially available and widely used. The amino acid sequences not highlighted are added as random sequences. The FLAG-HA peptide is used as an alternative to HJ-HA as an internal control for 5ADCSI assay.

In another aspect, bioassay system 10 includes a first set of control microspheres 18 attached to mouse polyclonal IgG (mIgG) to correct for background signal in individual specimens and a second set of control microspheres 20 configured to capture a synthetic peptide to normalize for well-to-well variations. These control microspheres work alongside the MagPlex® Monitoring Microspheres (IC45) that monitor assay and instrument performance. As described in the Materials and Methods section, HJ-HA peptide is added to each well as an internal control (150 pg/ml), and its signal is used in the normalization process. The statistical analysis section details how the MFI values are processed: first normalized based on IC45 readings for instrument variations, then the normalized HJ-HA values are used in relation to mIgG values, and finally each analyte's MFI is normalized using these controls. This strategic approach to normalization reduces variability, with average coefficient of variation values remaining below 3% across wells. This comprehensive control system significantly enhances the precision and reliability of the 5ADCSI assay for biomarker quantification.

In another aspect, a peptide (named NFL_UD1_2) corresponding to amino acids 310-362 of Neurofilament Light Chain (NFL) is synthesized and used as a calibrator. This peptide is based on the sequence presented by Shaw et al. in FIG. 1 of their publication (Shaw, G., et al., “Uman-type neurofilament light antibodies are effective reagents for the imaging of neurodegeneration,” Brain Communications, 2023, 5(2): fcad067), the entire disclosure of which is hereby incorporated by reference. The NFL_UD1_2 peptide contains both capture and detection epitopes for the Uman-type antibodies used in the 5ADCSI assay. As described in the Materials and Methods section, this synthetic peptide approach was chosen to overcome solubility inconsistencies observed with recombinant NFL proteins from different sources and to provide more accurate quantification given the range of molecular weights (48-70 kDa) for endogenous NFL. Due to these considerations, the concentration of NFL in samples is expressed in fmol/ml rather than pg/ml.

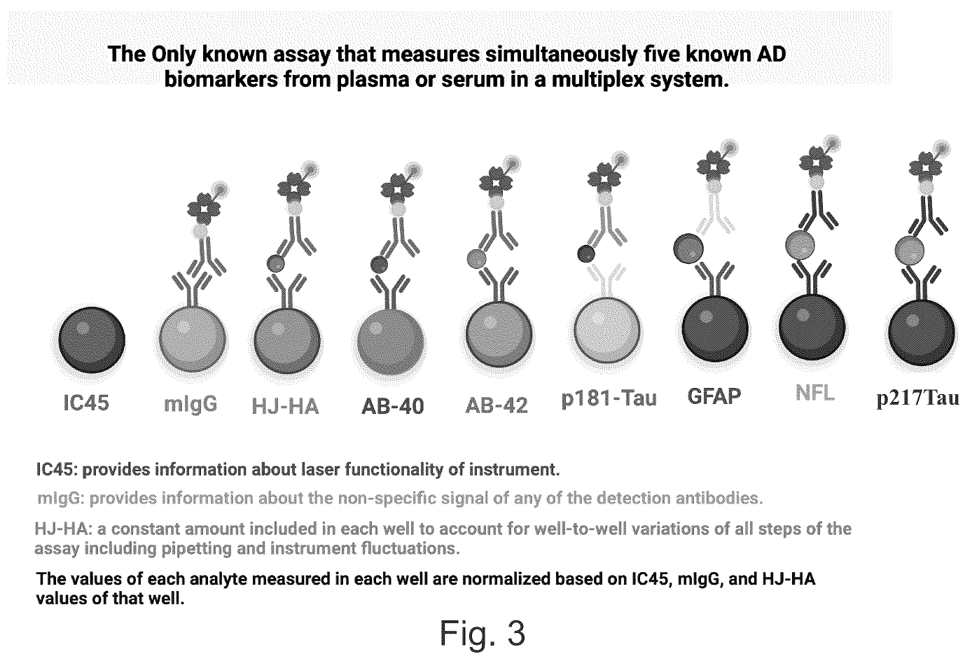

FIG. 3 depicts some of these microspheres. In a refinement, the NFL beads system detects and quantifies NFL protein fragments that are associated with Alzheimer's disease (AD). Data from the cerebral spinal fluid of normal control and AD subjects show this. The NFL beads system has excellent sensitivity to detect and quantify NFL in human plasma. High levels of NFL are an indicator of neuroaxonal damage regardless of the cause. Examples of neurodegenerative diseases where NFL has been shown to be a biomarker are AD, Parkinson's, amyotrophic lateral sclerosis (ALS), multiple sclerosis (MS), Huntington's disease, frontotemporal dementia (FTD), and traumatic brain injury (TBI). In a refinement, the GFAP beads detect and quantify glial fibrillary acidic protein (GFAP). GFAP as an emerging biomarker in brain and spinal cord disorders. A recent study (Nature Aging volume 4, pages 247-260 (2024)) shows that GFAP, NFL, and two other proteins are highly associated with all-cause dementia (ACD), Alzheimer's disease (AD), and vascular dementia (VaD).

In another aspect, the capture agent specific for Aβ40, Aβ42, pTau181, p217Tau, and Tau-441 are antibodies that specifically bind to Aβ40, Aβ42, pTau181, p217Tau, and optionally Tau-441 thereby allowing for selective detection and quantification of Aβ40, Aβ42, and pTau181, p217Tau (and Tau-441) levels in the presence of other analytes within the biological sample. In a refinement, the capture agent specific for NFL and GFAP are antibodies that specifically bind to NFL and GFAP thereby allowing for selective detection and quantification of NFL and GFAP.

In another aspect, the reporter molecules include a detection antibody that is biotinylated. In a refinement, the reporter molecules include a fluorescent moiety. Specifically, as detailed in the Materials and Methods section, the detection antibodies (including monoclonal antibody HJ5.1 for Aβ peptides, anti-Tau antibody 16-18 for tau proteins, MCA-1B1 for NfL, and 2.2B10 for GFAP) are biotinylated using a Pierce™ Antibody Biotinylation Kit according to the manufacturer's instructions. This biotinylation process enables the secondary detection step where Streptavidin R-phycoerythrin conjugate (SAPE) binds with high affinity to the biotin molecules. The phycoerythrin component of SAPE serves as the fluorescent reporter, emitting a detectable fluorescence signal upon excitation by the Luminex 200 analyzer's lasers. The intensity of this fluorescence (measured as Median Fluorescence Intensity or MFI) is directly proportional to the amount of captured analyte, enabling precise quantification of the Alzheimer's disease biomarkers in the biological sample.

In another aspect, the specific capture agents for Aβ40, Aβ42, pTau181, p217Tau, GFAP, and NFL are antibodies that specifically bind to Aβ40, Aβ42, pTau181, p217Tau, GFAP, and NFL, respectively, allowing for selective detection and quantification of these biomarkers in the presence of other analytes within the biological sample. As detailed in the Materials and Methods section, these capture antibodies are carefully selected commercial antibodies with demonstrated specificity: A040 antibody (11A50-B10, Biolegend), Aβ42 antibody (12F4, Biolegend), Phospho-Tau-Thr181 antibody (AT270, Invitrogen), p217Tau antibody (E9Y4S, Cell Signaling Technology), NFL antibody (MCA-6H63, Encorbio), and GFAP antibody (polyclonal, Proteintech). These antibodies are covalently coupled to distinct MagPlex magnetic microspheres using carbodiimide chemistry according to Luminex protocols. The specificity of each antibody pair was rigorously verified through multiple approaches, including testing with synthetic peptides, phosphatase treatment experiments, and specific immune depletion of human plasma samples, as demonstrated in FIG. 4. The validation experiments confirmed near 100% specificity for each capture/detection system with minimal cross-reactivity, ensuring accurate multiplex quantification of these important Alzheimer's disease biomarkers.

In another aspect, bioassay system 10 further includes a data analysis system 32 configured to perform the analysis set forth herein. For example, such systems can be configured to normalize signals using control microspheres and to calculate ratios between different analytes, wherein the ratios include at least one of: Aβ42/Aβ40, p217Tau/Aβ42, and p181Tau/Aβ42. In a refinement, the data analysis system configured to calculate a p217Tau/Aβ42 ratio from quantified analytes, wherein the p217Tau/Aβ42 ratio provides enhanced diagnostic accuracy for distinguishing Alzheimer's disease from cognitively normal individuals.

In another aspect, a method for multiplexed detection and quantification of multiple analytes in a biological sample using the system set forth above is provided. The method comprises mixing the biological sample with a plurality of sets of color-coded microspheres, including a first set of microspheres distinguishable by a first unique color code generated by internal dyes, where each microsphere in the first set is coated with a specific capture agent that binds to Aβ40; a second set of microspheres distinguishable by a second unique color code, each coated with a specific capture agent that binds to Aβ42; a third set of microspheres distinguishable by a third unique color code, each coated with a specific capture agent that binds to p181Tau or p217Tau; a fourth set of microspheres distinguishable by a fourth unique color code, each coated with a specific capture agent that binds to glial fibrillary acidic protein (GFAP); and a fifth set of microspheres distinguishable by a fifth unique color code, each coated with a specific capture agent that binds to Neurofilament Light Chain (NFL). The method further includes allowing specific binding between analytes in the biological sample and their respective capture agents on the microspheres, introducing reporter molecules that bind to the captured analytes, wherein the reporter molecules emit a detectable reporter fluorescence upon excitation, and passing the microspheres through a flow cell. The method then includes exciting and reading the fluorescence of each internal dye and the detectable reporter fluorescence indicative of analyte binding, followed by identifying each of the sets of microspheres by its associated unique color code and quantifying the bound analytes based on the detectable reporter fluorescence.

In another aspect, the method further comprises adding a first set of control microspheres attached to mouse polyclonal IgG to correct for background signal associated with individual specimens, and adding a second set of control microspheres configured to capture a synthetic peptide to normalize for well-to-well variations.

In another aspect, the method further comprises calculating at least one ratio selected from the group consisting of the Aβ42/Aβ40 ratio, the p217Tau/Aβ42 ratio, and the p181Tau/Aβ42 ratio, wherein these ratios provide enhanced diagnostic accuracy for distinguishing between cognitively normal individuals, individuals with mild cognitive impairment, and individuals with Alzheimer's disease.

The following examples illustrate the various embodiments of the present invention. Those skilled in the art will recognize many variations that are within the spirit of the present invention and the scope of the claims.

### Materials and Methods

### Clinical and Neuropsychological Assessment of all Participants

For the validation studies presented below we used matched CSF, plasma and serum from University of Southern California Alzheimer's Disease Research Center (USC ADRC). The use of the specimens for this study was approved by the USC Institutional Review Board (IRB #: HS-24-00439). The enrollment, collection, diagnoses based on history, physical examination, cognitive and functional assessment, and PET imaging were completed by the USC Alzheimer's Disease Research Center (USC ADRC) per National Alzheimer's Coordinating Center guidelines and the USC ADRC protocol and are described in the Supplementary Materials and Methods.

### Plasma and Serum Stabilization

Frozen human CSF, plasma and serum samples were thawed and supplemented with protease and phosphatase inhibitors (Roche cOmplete, Mini, EDT A-free, Sodium fluoride 4.2 μg/ml, Sodium orthovanadate 30 μg/ml, beta-glycerol-phosphate 40 μg/ml, and Sodium pyrophosphate 40 μg/ml). Plasma or serum samples were sonicated for 5 min at 4° C. and centrifuged at 10000 g for 5 min at 4° C. The protein concentration of samples was determined by the pierce quantitative colorimetric peptide assay kit using Albumin Standard (ThermoFisher, Cat #:23209) as standard curve. The concentrations were adjusted to 20 mg/ml plasma protein with dilution buffer containing 1×PBS, 200 mM EPPS, 250 GuHCl, 0,225% CHAPS, and 2 mM EDTA (pH of 7.4). To inhibit non-specific background, the following were added to each well and incubated it at 4° C. for 2 hours: 2 μg mouse IgG (Bio-Rad PMP01X), 0.3 μg IG1_k (Bio legend 400102), and 20 μg Immunoglobulin Inhibiting Reagent (IIR, BIOIVT),

### Luminex Assay

We selected the capture sandwich immunoassay (CSI) xMAP technology, utilizing Luminex instruments (https://www.luminexcorp.com). The “x” in xMAP signifies the biomarker or disease panel to be tested, while MAP stands for Multi Analyte Profiling (xMAP Cookbook. Ed4.WR, Luminex Corp.) The xMAP technology, a widely used methodology, offers commercially available kits for a variety of analytes. Our assay utilizes fundamental xMAP components and existing antibodies.

### Coupling of Antibodies to the Carbodiimide-Modified Magplex Magnetic Microspheres

MagPlex magnetic microspheres (Luminex; product #MC10012-01, #MC10019-1, #MC10025-1, #MC10038-1, #MC10044-1, and #MC10056-1) coupling reactions were carried out using antibodies [(HA Antibody/16B12, Biolegend Cat #:901502), (Aβ40/11A50-B10, Biolegend Cat #: 805402), (Aβ42/12F4, Biolegend Cat #: 805502), (Phospho-Tau-Thr181/AT270, Invitrogen, Cat #: MN1050), p217Tau ((E9Y4S, product #64265SF, Cell Signaling Technology), NfL antibody (MCA-6H63 for capturing Antibody, Encorbio, Cat #MCA-6H63), GFAP polyclonal Antibody, Proteintech, Cat #16825-1-AP]. The “Carbodiimide Coupling Protocol for Antibodies and Proteins” in the Luminex xMAP Cookbook v4.0 was followed for the standard coupling of peptides to Magplex microspheres (xMAP Cookbook. Ed4.WR, Luminex Corp.). Briefly, 2.5×106 beads were washed with dH₂O and activated for 20 minutes in the dark using end-over-end mixing in 80 μl 0.1 M Sodium Phosphate (monobasic), pH 6.2, plus 10 μL of 50 mg/mL 1-ethyl-3-[3-dimethylaminopropyl]carbodiimide hydrochloride (EDC; ThermoFisher Scientific) and N-hydroxysulfosuccinimide (Sulfo-NHS; ThermoFisher Scientific). Beads were washed 3 times with 250 ul 50 mM MES buffer, pH 5.0. The activated beads were then resuspended in the 100 μl MES buffer pH 5.0, and 25 g of each antibody/2.5×106 beads were added to the resuspended beads mixture. Antibodies were dialyzed using Slide-A-Lyzer MINI Dialysis Device, 10K MWCO with 1×PBS pH 7.4 before using for coupling (ThermoFisher Scientific). Then using MES buffer, the total volume of the reaction was brought to 500 μL. The coupling reaction was performed for 2 hours on the rotary shaker in the dark. The antibody-coupled beads were then washed with blocking buffer (1×PBS+0.1% BSA+0.02% Tween-20+0.05% sodium azide, pH 7.4) and were further blocked for 30 minutes. The coupled and blocked beads were resuspended in 500 μL storage buffer (1×PBS+1% BSA+0.02% Tween-20+0.05% sodium azide, pH 7.4) and stored at 4° C. in the dark. The beads were counted with a hemocytometer. All procedures were performed at room temperature. Finally, coupling confirmation was carried out using a PE-labeled anti-mouse IgG detection antibody.

### Biotinylation of Detection Antibodies

Biotinylation of the secondary antibodies (monoclonal antibody HJ5.1, a generous gift from Dr. D.M. Holtzman²), (anti-Tau, 16-18, Biolegend, Cat #:806501), (NfL monoclonal MCA-1B1, Encorbio, Cat #MCA-1B11), and GFAP Monoclonal Antibody (2.2B10), Thermofischer, Cat #13-0300] was carried out using a Pierce™ Antibody Biotinylation Kit following the manufacturer's instructions (ThermoFisher Scientific). Briefly, 100 g of the dialyzed antibody with 1×PBS were mixed with 3.1 μl of 8.5 mM NHS-PEG4-Biotin by gently pipetting up and down and incubated the reaction at room temperature for 30 minutes. Then the desalting procedure was performed using Zeba Spin Desalting Column, and biotinylated antibodies were stored at 4° C.

### Plasma Immune Depletion

Immune depletion of plasma Aβ40, Aβ42, Tau12-pT181, p217Tau, NfL, and GFAP were performed using biotinylated specific antibodies of each analyte (biotinylated mouse IgG as a control) and Pierce streptavidin magnetic beads (Thermo Scientific™, Cat: 88816). Briefly, biotinylated Aβ40, Aβ42, Phospho-Tau/Thr181, Phospho-Tau/Thr217, NfL, GFAP or biotinylated mIgG (as negative control) were added individually to the 2 mg plasma samples. Samples were incubated on rotary shaker at 4° C. for 10 hours. Streptavidin magnetic beads were added to the sample and incubated for an additional 2 hours at 4° C. After the separation of magnetic beads using Magjet rack (Thermo Scientific), each analyte was quantified by 5ADCSI.

### Phosphatase Experiment

For each p181Tau and p217Tau phosphatase reaction 400 ng of a human postmortem brain extract of a subject with AD were treated either with 400 units (1 microliter) of lambda protein phosphatase (New England BioLabs, Catalog #P0753S) or 1 microliter water (control)according to the manufacturer conditions and supplied buffer for 30 minutes at 30° C. Each reaction was supplemented with 20 mg/ml BSA, and phosphatase was deactivated at 60° C. for 15 minutes. The p181Tau and p217Tau in each reaction was determined using 5ADCSI assay with corresponding p181Tau and p217Tau microspheres.

### Luminex Assay

With some modifications, the “Indirect (serological) immunoassay” of Luminex xMAP Cookbook v 4.0 (Stephen Angeloni, S.D., Dunbar, Sherry, Stone, Valerie, Swift, Sarah, 2018. xMAP Cookbook: A Collection of Methods and Protocols for Developing Multiplex Assays With xMAP Technology, Luminex 4th edition) served as the foundation for the Luminex single plex or multiplex assay (xMAP Cookbook. Ed4.WR, Luminex Corp.). We pooled human plasmas from 10 individuals with ages from 30-70 (purchased from Innovative Research company) as a quality control in each plate. To each well, 150 μg/ml of HJ-HA peptide was added as an internal control. The amounts of plasma and serum used equals to approximately to a dilution of 3-folds, and for CSF equals to approximately a dilution 10-folds of original samples. All samples were analyzed in triplicates. For one well, 2 mg (100 ul) of the pre-prepared plasma, serum, or CSF (25 ug in 2 mg BSA) was mixed with 25 μl of 2 μg/ml corresponding biotinylated detection antibodies, and antibody coupled beads mixture (prepared by adding approximately 1500 beads per region of IC45 MagPlex® Monitoring Microspheres, mouse IgG, HJ-HA, AB40, AB42, p181Tau, NfL, and GFAP in a flat-bottomed 96-well plate (Corning, MA, USA). The plate was incubated for 2 hours on a microplate shaker at 800 rpm at room temperature, then washed three times with 100 μl wash buffer (1×PBS, 1% BSA, 0.05% sodium Azide, pH 7.4) using magnetic 96-Well separator (ThermoFisher Scientific). Beads were resuspended in 50 ul/well assay buffer and 50 μl/well of 4 ug/ml Streptavidin R-phycoerythrin conjugate (SAPE) (Agilent Technologies, Part No: PJRS34-1) and incubated for another 30 minutes on a microplate shaker at 800 rpm at room temperature. After washing as above, the beads were resuspended in 100 μl/well of assay buffer and analyzed using a Luminex 200 analyzer with a minimum of 50 beads acquired for each bead region. The mean fluorescence intensity (MFI) from each well was calculated by using Xponent Software, version 4.3 (Luminex, Austin, TX, USA).

### Luminex 200 Instrument Calibration and Verification

The Luminex 200 system (Luminex Corporation, Austin, TX, USA) was employed for this study, consisting of a Luminex 200 instrument and associated software (Luminex xPONENT) for data acquisition. The instrument was calibrated as recommended by the manufacturer on a weekly basis using the calibration kit (Cat #: LX2R-CAL-K25, Luminex Inc.). Before each measurement, the instrument underwent a manufacturer recommended verification using a verification kit (Cat #: LX2R-PVER-K25, Luminex Inc.) to confirm proper instrument functionality and alignment with current calibration settings.

### Statistical Analysis

All data were collected from experiments performed in triplicates. We wrote R (R Core Team, 2024)³³ code to automate data processing steps described below and conversion of MFI values to concentrations using standards run in each plate to generate calibration curves. First, the MFI (Median Fluorescence Intensity) of each analyte including mIgG were normalized based on IC45 MFI for instrument reading variations. The average % CV of instrument reading variations is less than 0.7% for each plate (inter plate), and less than 0.65% across 100 plates (intra plate). Second, for each well the normalized MFI of HJ-HA was subtracted from the normalized MFI of mIgG in the same well. The average % CV of well-to-well of mIgG in each plate is less than 3%. Third, the MFI of each analyte is normalized based on the MFI of HJ-HA from step 2. The average well-to-well % CV (intra plate) of HJ-HA MFI in each plate is less than 2.19% (Supplementary FIG. 4). The quality control (QC) of each individual plate is assessed by three means: (1) the expected MFI value of the pooled plasma (run at the first three and last three wells on each plate), (2) by the expected value of average MFI of HJ-HA of the same amount spiked in each well in every plate, and (3) by the MFI values of the calibrators (standard curve) for each analyte. To avoid inaccuracies associated with insolubility of recombinant Tau proteins and in vitro phosphorylation efficiency issues, we used synthetic peptides (FIG. 4B) as calibrators. We then used fmol/ml instead of pg/ml for quantification of p-Tau proteins. The MW range of Tau proteins in plasma are estimated to be 48-67 kDa¹⁵. To accurately estimate the concentration of p181Tau and p217Tau we utilized the synthetic peptide Tau12pT181 and Tau12pT217 which are 100% phosphorylated and have a molecular weight (MW) of 4.01, and 4.23 kilodaltons (kDa), respectively as the calibrators. Due to the significant differences of the molecular weights of endogenous Tau proteins (48-67 kDa) to the synthetic phospho-Tau used here as calibrators the plasma p181Tau and p217Tau levels are expressed as fmol/ml.

The endogenous NfL exists in a range of molecular weights (48-70 kDa), We also encountered solubility inconsistencies in using recombinant NfL from different sources. To be more accurate and avoid solubility inconsistencies of recombinant NfL in standards, we used a synthetic peptide NfL_UD1_2 (MW: 6.08 kDa, Supplementary Materials and Methods), which contains both capture and detection Uman-type antibody epitopes for NfL¹⁶. Thus, we express the concentration of NIL in fmol/ml.

The final analyte concentrations in plasma, serum and CSF were calculated by multiplying the measured concentrations by the dilution factors (3-folds for plasma and serum, and 10-folds for CSF).

Data visualization and statistical analysis was performed using GraphPad Prism 10 (GraphPad Software, San Diego, CA, USA) and R studio software version 4.3. All data are presented as mean±standard deviation (SD) or mean±95% confidence interval (CI). Mann-Whitney nonparametric paired samples t-tests were used to assess the significance of differences between two groups. Pearson's correlation coefficients were used to assess association of analytes values between CSF and serum or plasma groups.

The diagnostic accuracy (sensitivity and specificity) of the 5ADCSI in differentiating CN versus MCI or AD was assessed by logistic regression and receiver-operating characteristic (ROC) curves constructed from the logistic scores. The areas under the curves (AUCs) for analytes and corresponding ratios were calculated with corresponding standard errors, 95% CI and p-values.

### Results

### Development of 5ADCSI

We built 5ADCSI to achieve three key objectives: exerting control over assay components (specifically the capacity for multiplexing), enhancing sensitivity, accuracy, and cost savings. In addition to the microspheres with capturing specificity to target analytes (Aβ40, Aβ42, p181Tau, p217Tau, NfL, and GFAP), we have incorporated three additional microspheres into the assay to further improve accuracy, reproducibility, and sensitivity: 1. MagPlex® Monitoring Microspheres (IC45, Luminex Inc.) to monitor assay and instrument performance, including fluctuations and user errors. 2. A microsphere attached to polyclonal mouse Immunoglobulin G (mIgG) antibodies to assess the background of each sample. 3. A microsphere attached to the HA (hemagglutinin) antibody to quantify a synthetic peptide (HJ-HA, see Supplementary Materials and Methods for peptide sequence) added at a constant amount (150 pg/ml) for normalizing target analytes. Because p181Tau and p217Tau will compete for similar Tau proteins, they cannot be used in a multiplex assay simultaneously. Therefore, the 5ADCSI can be run in two different formats with either p181Tau or p217Tau with other 4 analytes.

Specificity/Cross-Reactivity and Sensitivity of 5ADCSI for Quantification of Aβ40, Aβ42, p181Tau, p217Tau, NfL, and GFAP in Plasma

We investigated the specificity of capture and detection of antibodies for Aβ40 and Aβ42, p181Tau, p217Tau, NfL, and GFAP microspheres: (A) using synthetic peptides (Aβ40 and Aβ42, p181Tau, p217Tau and NfL), and recombinant gst-GFAP (Glutathione S transferase-GFAP fusion recombinant protein) to demonstrate antibody-target capture specificity (FIG. 4A), (B) depletion of each analyte with corresponding antibodies to show specific recognition in plasma (FIG. 4D). Antibodies to capture Aβ40 and Aβ42 recognize C-terminal portions of each peptide. The detection antibody (monoclonal antibody HJ5.1) recognizes an epitope in the N-terminal region of both Aβ40 and Aβ42 as well as HJ-HA peptide (see Supplementary Materials and Methods).

For p181Tau and p217Tau we designed and synthesized two identical peptides for each (FIG. 4B), except for a single difference where phospho-threonine was used instead of threonine in the positions corresponding to T181 and T217 in Tau (Uniprot ID P10636-8). Each peptide consists of two epitopes with extended amino acids linked together. The epitope recognized by the capture antibody, the AT270 mouse monoclonal antibody also known as N-terminal p181-Tau (MN1050; Invitrogen, Waltham, MA, USA) includes T181 or pT181. This antibody has been characterized for specific capture of p181Tau by Karikari et al. The N-terminal epitope is recognized by a detection antibody composed of N-terminal 12 amino acids of Tau proteins. The same strategy was used for the p217Tau peptide. The capture antibody for p217Tau is a rabbit monoclonal p217Tau phospho-specific antibody (E9Y4S). Utilizing the synthetic phosphorylated Tau12-pTau181 and Tau12-pTau217 peptides offers distinct advantages over recombinant Tau protein. The synthetic peptide ensures full phosphorylation of the threonine 181 and 217 residues, a critical aspect often compromised when recombinant proteins are phosphorylated in vitro. Additionally, unlike recombinant proteins which may suffer from solubility issues, the synthetic peptide exhibits consistent solubility, ensuring day-to-day calibration consistency.

Data in FIG. 4A indicate that the Aβ40 and Aβ42 capture microspheres identify their corresponding peptides specifically, and the p181Tau and p217Tau capture microspheres identify phosphorylated Tau12-pTau181, p217Tau peptides, respectively, but not non-phosphorylated Tau12-T181 and Tau12-T217 Tau peptides at various concentrations. For NfL, we assessed the specific peptide encompassing amino acids 310-362 (NfL Rod region, we named it NfL_UD1_2 peptide in Supplementary Materials and Methods) to a C-terminal peptide of NfL. The NfL_UD1_2 peptide contains epitopes for capture and detection antibodies analogous to the Uman-type antibodies used for quantification of NfL from plasma and CSF¹⁷. For GFAP, we used the same capture antibody that was characterized by Fazeli et al¹⁸ as capture antibody in quantification of GFAP in human plasma, which was directly compared to SIMOA GFAP quantification system¹⁸. We used recombinant gst-GFAP against gst protein for specificity test shown in FIG. 4A. The data in FIG. 4A indicate near 100% specificity for Aβ40, Aβ42, p181Tau, p217Tau, NfL, and GFAP capture/detection system for their corresponding peptides.

We further examined the specificity of p217Tau and p181Tau capture antibodies for endogenous phospho-Tau proteins. We treated human postmortem brain extract of a subject with AD with lambda protein phosphatase and asses the levels of p181Tau and p217Tau to not phosphatase treated extract (FIG. 4C). Phosphatase treatment of the brain extract significantly reduced the detection of p181Tau and p217Tau.

To further investigate the specificity of each Aβ40, Aβ42, p181Tau, p217Tau, NfL, and GFAP capture/detection systems in plasma, we depleted each analyte with their specific capture antibodies from plasma and compared it to plasmas depleted with a mIgG (FIG. 4D). The depletion with analyte-specific antibodies results in significant reduction of their specific MFI signal compared to the mIgG-depleted plasma (FIG. 4D), providing further evidence for specificity of capture and detection system for these proteins in plasma.

Experiments described below were performed in two different multiplex modes with Aβ40, Aβ42, NfL, GFAP with either p181Tau or p217Tau as the fifth analyte.

The 5ADCSI performed equally well in single versus 5-plex mode in quantifying each biomarker (Supplementary FIG. 1). We calculated the limit of blank (LoB), limit of detection (LoD), and estimated the limit of quantification (LoQ) according to Armbruster and Pry (2008) (Supplementary Table 1)¹⁹. We used ten independent runs of eleven concentrations of standard peptides (0-1000 μg/ml) in different days for LoQ estimation (Supplementary Table 1). The estimated LoQs are 3, 2.1, 2.6 pg/ml for Aβ40, Aβ42, and GFAP, respectively, and 0.05, 0.23, 0.23 fmol/ml for NfL, p181Tau and p217Tau, respectively. The inter assay CVs for each concentration of various analytes is 2-4.5%, and intra assay of all analytes at various concentrations is 1.9-4.6%. These data indicate excellent precision for 5ADCSI (Supplementary FIG. 3). The recovery for each analyte at the LoQs were 90-110%. The lowest concentrations of Aβ40, Aβ42, p181Tau, p217Tau, NfL and GFAP measured in this report (see Table 1), and other reports using different technologies^{2,4,5,13,20-22}, are significantly higher than the LoQ for each analyte.

The dynamic range of quantification for each analyte are shown in Supplementary Table 1 and Supplementary FIG. 2.

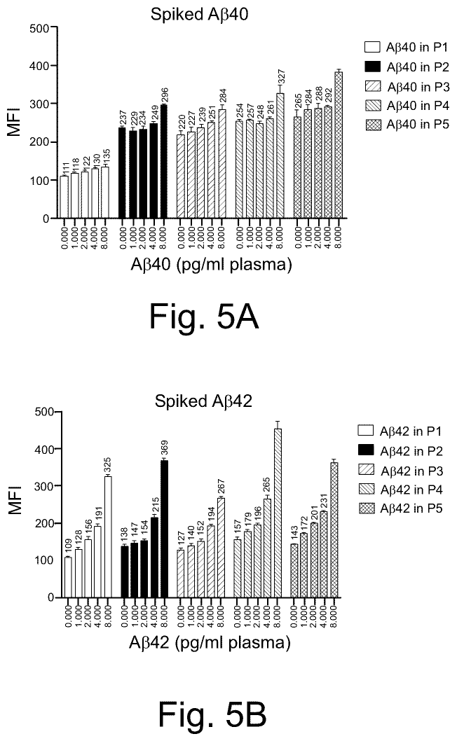

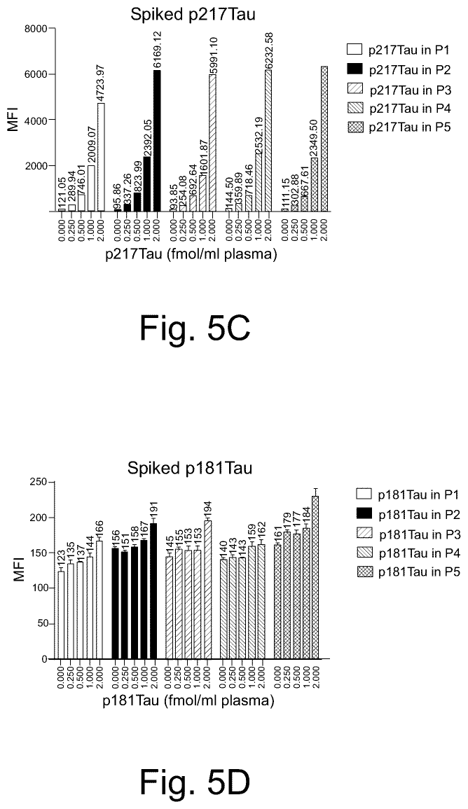

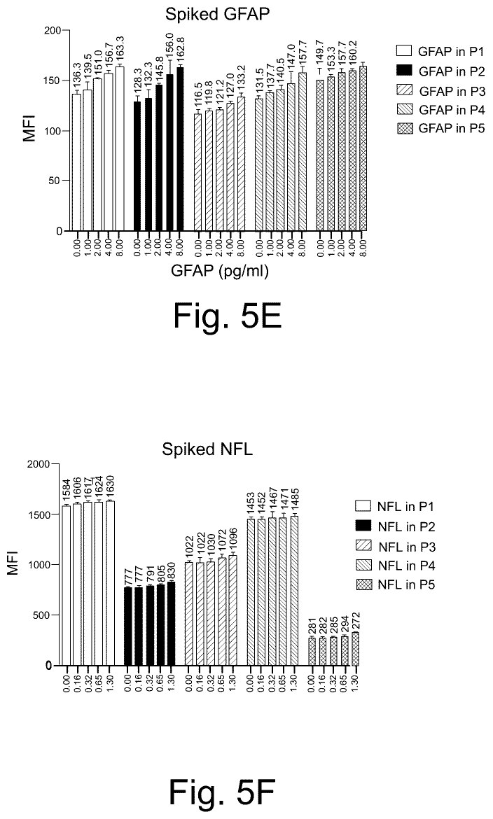

As the LoQs were determined with BSA as background matrix, we assessed the detection sensitivity in plasma and the validity of LoQs of Aβ40, Aβ42, p181Tau, p217Tau, NfL, and GFAP in plasma or serum by 5ADCSI, we spiked 0, 1, 2, 4, or 8 μg of each analyte in 20 mg/ml of five plasmas or serums from different individuals (FIG. 5). The pg/ml values for p181Tau, p217Tau and NfL were converted to fmol/ml. At various levels of endogenous amounts of each analyte (Aβ40, Aβ42, p181Tau, p217Tau, NfL, and GFAP). Consistently all analytes exhibited significant enhancement of MFI signal starting at or below their LoQs (FIG. 5).

Quantification of Aβ40, Aβ42, p181Tau, p217Tau, NfL, and GFAP from Matched CSF, Plasma and Serum of CN, MCI, and AD Subjects Using 5ADCSI

We quantified Aβ40, Aβ42, p181Tau, p217Tau, NfL, and GFAP levels in matched CSF, plasma, and serum of subjects clinically diagnosed as CN, to subjects diagnosed with MCI or AD in a cross sectional study. Demographic details and summary statistics are presented in Table 1. CN and MCI participants (n=35 for CN, n=17 for MCI) included serum samples and their corresponding matched CSFs. The AD group (n=11) comprised of 5 plasma and 6 serum samples with corresponding matched CSFs. The average ages for CN, MCI, and AD cohorts were 67.8±7.98, 69.82±9.82 and 65.81±15.13, respectively. Gender distribution was 14/21 (male/female) for CN, 11/6 for MCI, and 6/5 for AD cohorts. The means of the Clinical Dementia Rating-Sum of Boxes (CDR-SB) scores are CN group: 0.2±0.35, MCI group: 1.4±0.7, and AD group: 5.63±4.38 (AD). The Montreal Cognitive Assessment (MoCA) scores averaged 26.8±2.28 (CN), 21.12±3.28 (MCI) and 15.50±6.36 (AD).

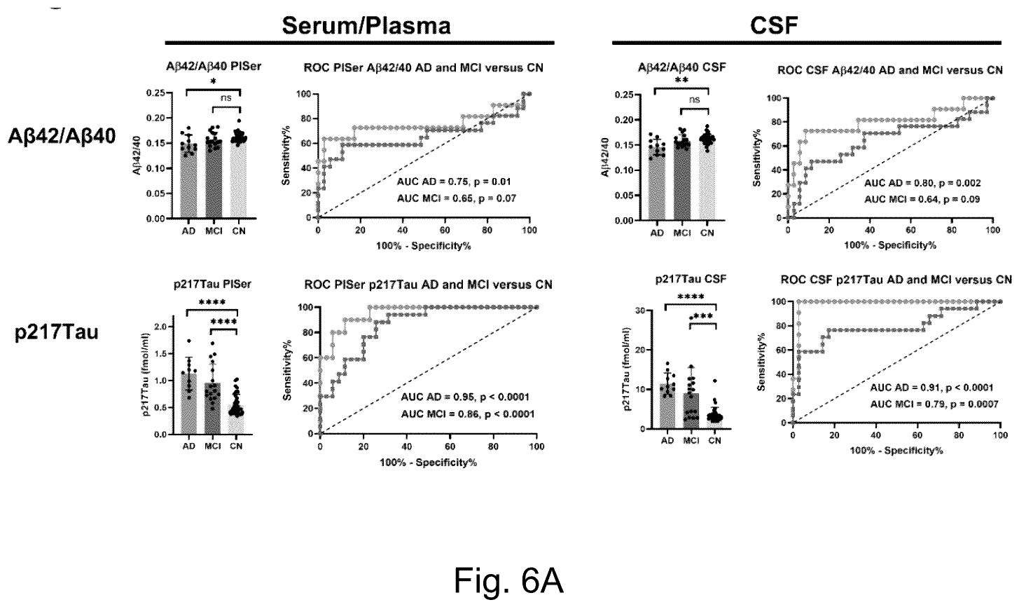

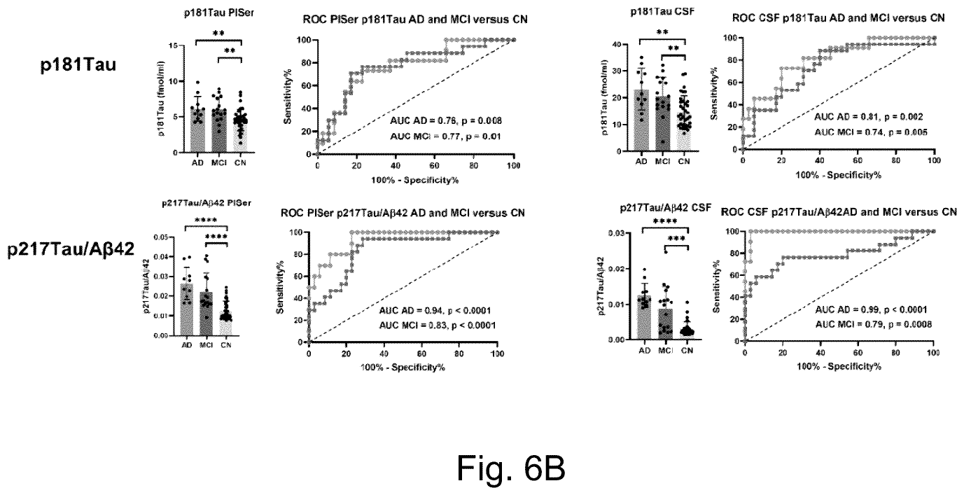

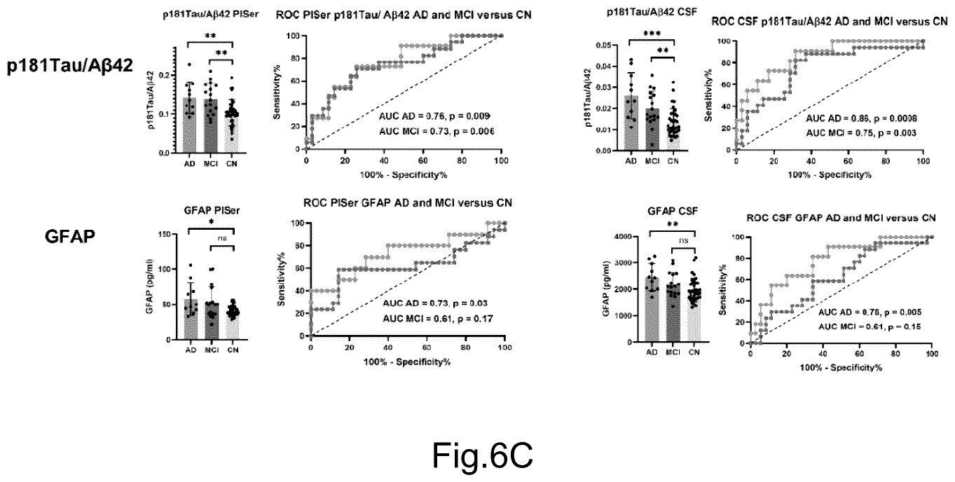

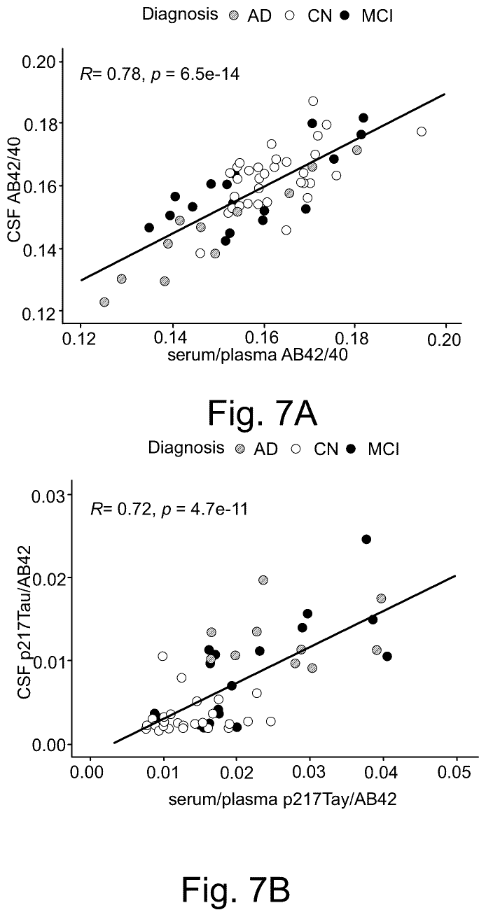

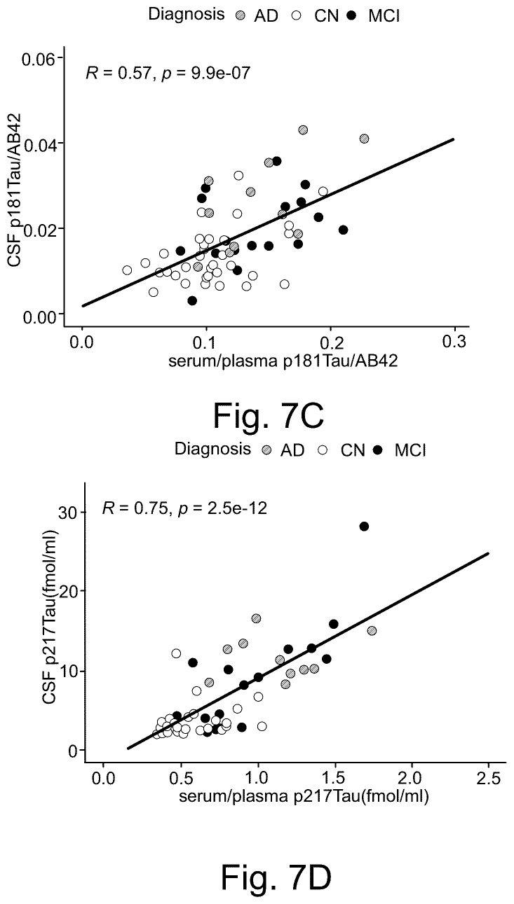

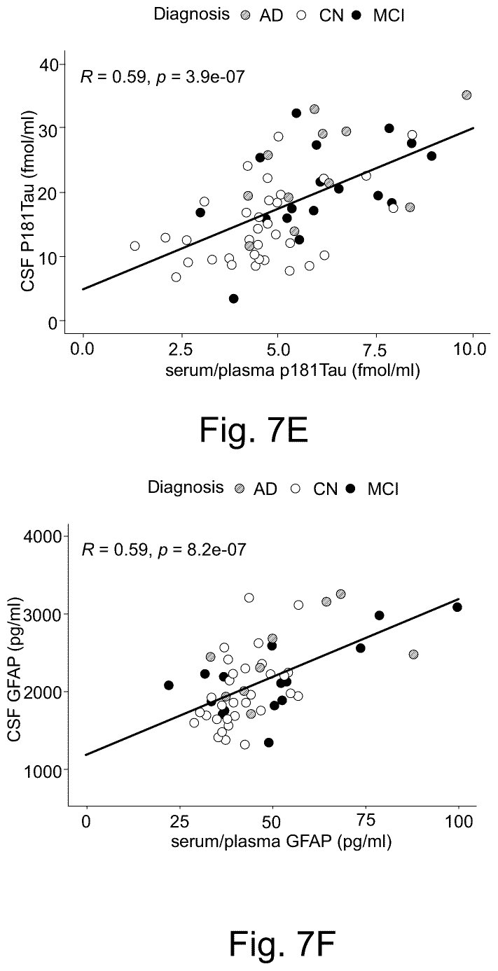

Table 1, FIG. 6 and Supplementary FIG. 4 illustrate the mean pg/ml for Aβ40 and Aβ42, GFAP and fmol/ml for p181Tau, p217Tau and NfL) and 95% confidence interval (CI) of quantified biomarkers. In CSF, the mean Aβ42/40 ratio of AD group [0.146 (0.136-0.156)] is 9.8% lower than the CN [0.162 (0.159-0.166). In serum/plasma, the mean Aβ42/40 ratio of AD group [0.149(0.137-0.161)] is 8.3% lower than the CN [0.162 (0.159-0.165). The AUC of the ROC curve of AD versus CN for the mean Aβ42/40 ratio in CSF is 0.8 and MCI versus CN is 0.65 (FIG. 6). Matching plasma and serum ratios of Aβ42/40 are [0.162 (0.159-0.165)] for CN and 0.157 (0.150-0.164) for MCI, and [0.149 (0.137-0161)] for AD, with an AUC of 0.75 for AD versus CN and 0.64 for MCI versus CN (FIG. 6). Pearson correlation of Aβ42/40 ratios between CSF and plasma/serum values is significant (r=0.77, p=0.00054, FIG. 7).

For p217Tau, the mean values (fmol/ml, 95% CI range) in CSF's CN group increases from 3.61 (2.97-4.26) to 9.01 (5.67-12.4) in the MCI group (AUC=0.79), and to 11.5 (9.66-12.3) in AD group (AUC=0.91). Corresponding values in plasma/serum increase from 0.55 (0.49-0.62) for CN to 0.95 (0.77-1.13) for MCI (AUC=0.86), and to 1.13 (0.91-1.35 for AD (AUC=0.95). The correlation of p217Tau between CSF and plasma/serum values (FIG. 7) (r=0.86, p=3.9xe⁻⁵).

We calculated mean (95% CI range) of p217Tau/Aβ42 ratios, which increase from 0.003 (0.002-0.004) in the CSF of the CN group to 0.009 (0.005-0.012) in the CSF of the MCI group (AUC=0.79), and to 0.013 (0.010-0.015) for the AD group (AUC=0.99). Corresponding values in plasma/serum increase from 0.013(0.011-0.014) for CN to 0.022 (0.017-0.027) for MCI (AUC=0.83), and to 0.027 (0.020-0.032) for AD (AUC=0.94). A significant Pearson correlation (r=0.83, p=0.0001) is observed for p217Tau/Aβ42 ratios between CSF and plasma/serum values (FIG. 7).

The data for p181Tau and p181Tau/Aβ40 in CSF and matched plasma/serum follow the same trend as p217Tau and p217Tau/Aβ42 (Table 1), though while significant, but with lower AUCs (FIG. 6). Importantly, the values for p181Tau and p181Tau/Aβ40 in CSF correlate well with values in matched plasma/serum with correlation coefficients of 0.63 and 0.67, respectively, with statistically significant p values (FIG. 7).

Calculated mean (95% CI range) of GFAP increases from 1982 (1831-2132) pg/ml in the CSF of the CN group to 2170 (1932-2409) pg/ml in the CSF of the MCI group, with AUC=0.61, and to 2451 (2104-2798) pg/ml in the CSF of the AD group with AUC=0.78. Corresponding values in matched plasma/serum increase from 41.5 (39-44) pg/ml for the CN group to 52.8 (41.1-64.5) pg/ml for the MCI group, with AUC=0.61, and to 58 (41.2-74-9) pg/ml for the AD group with AUC=0.73. The Pearson correlation (R=0.59, p=8.2xe⁻⁷) is significant for GFAP ratios between CSF and plasma/serum values (FIG. 7).

Calculated mean (95% CI range) of NfL decreases from 4.7 (3.8-5.7) fmol/ml in the CSF of the CN group to 0.1 (0.7-1.3) fmol/ml in the CSF of the MCI group, with AUC=0.88, and to 1.6 (1.1-2.1) fmol/ml in the CSF of the AD group with AUC=0.82 (Table 1 and Supplementary FIG. 5). Corresponding values in plasma/serum increase from 60.5 (29.5-91.5) fmol/ml for CN to 75.1 (13.2-137) fmol/ml for MCI, with AUC=0.5, and to 68 (−19.5-155) fmol/ml in AD group with AUC=0.5 (Table 1 and Supplementary FIG. 5). The NfL values in plasma and serum vary significantly among subjects, and the correlation (R=−0.26, p=0.84). between CSF and plasma and serum values are not significant (Supplementary FIG. 5).

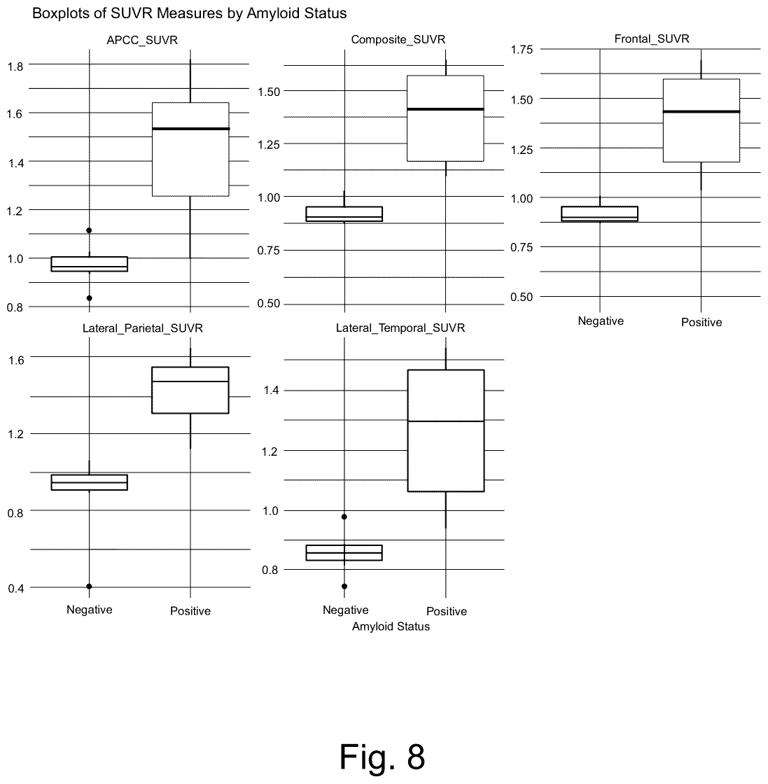

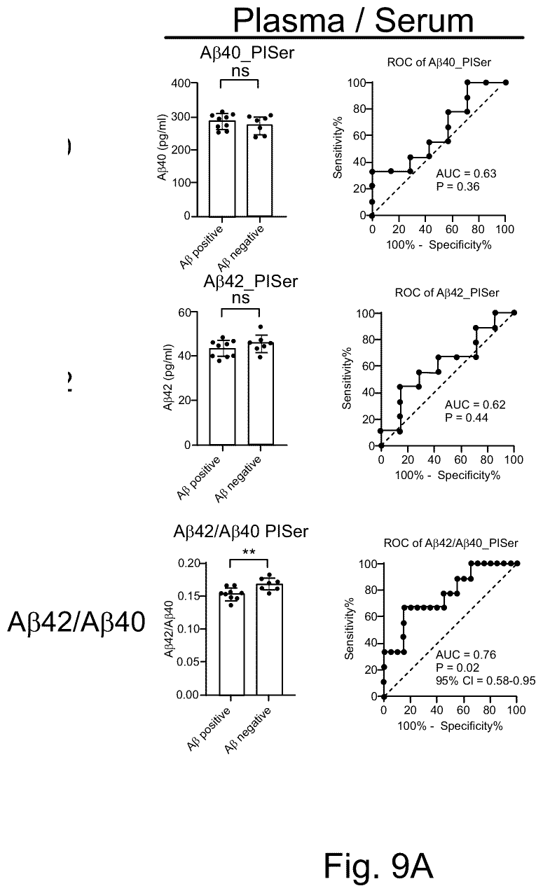

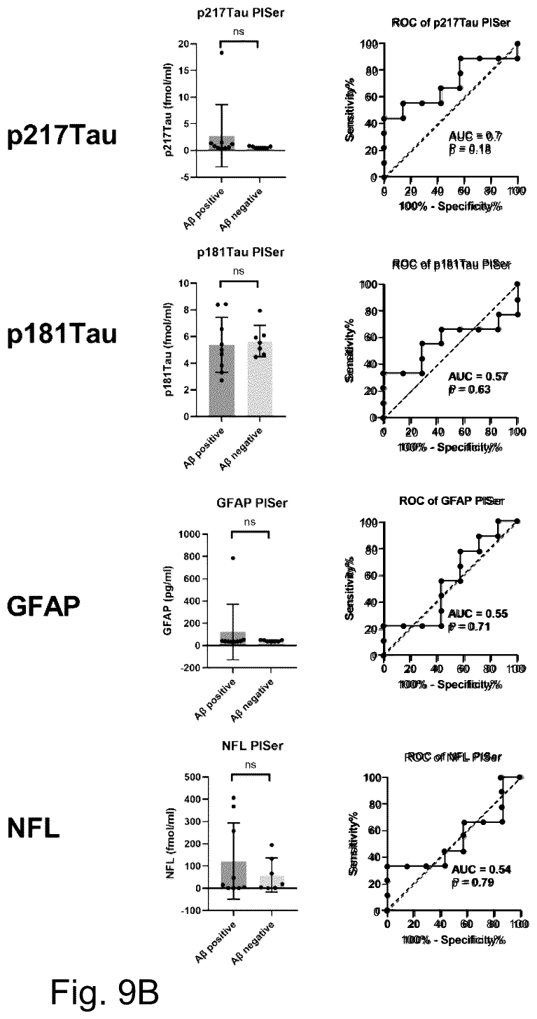

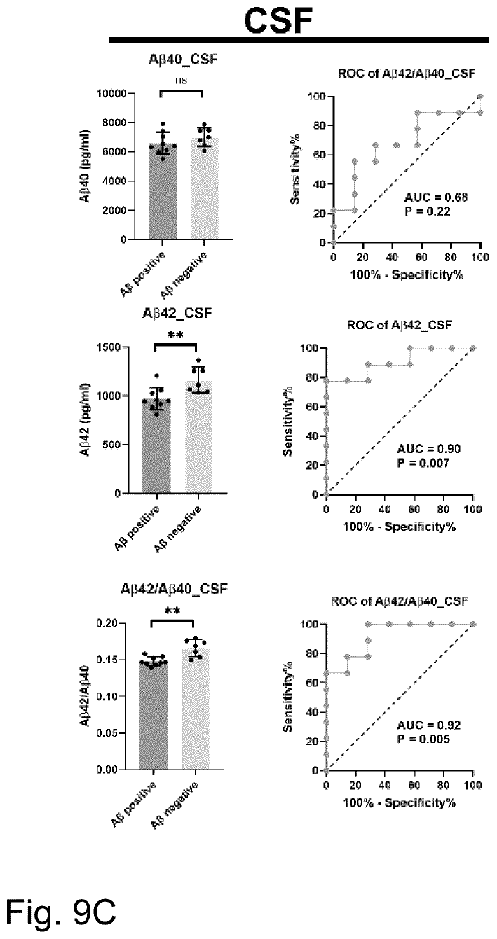

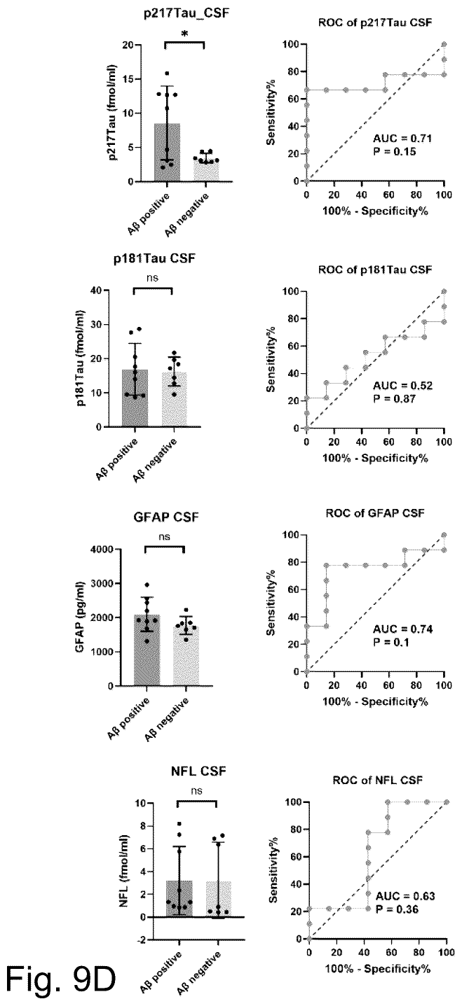

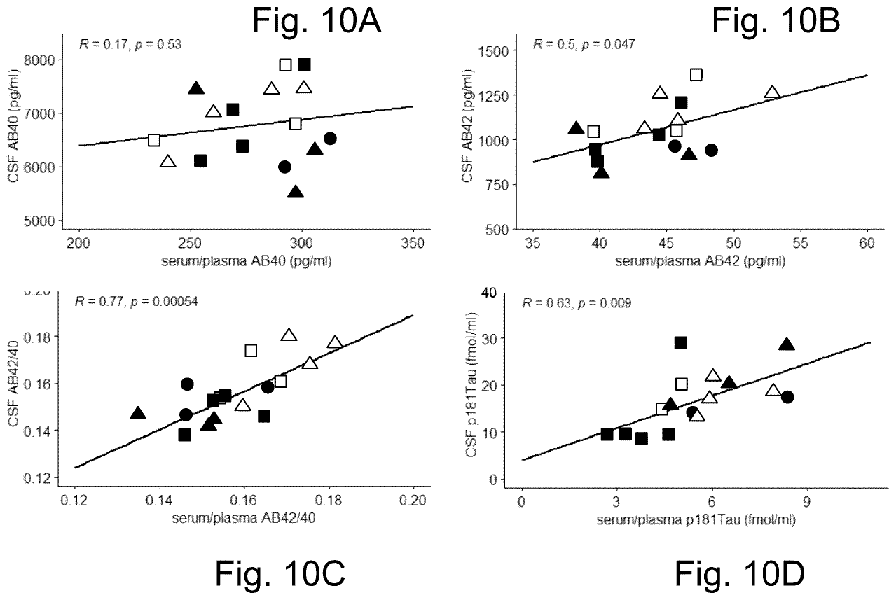

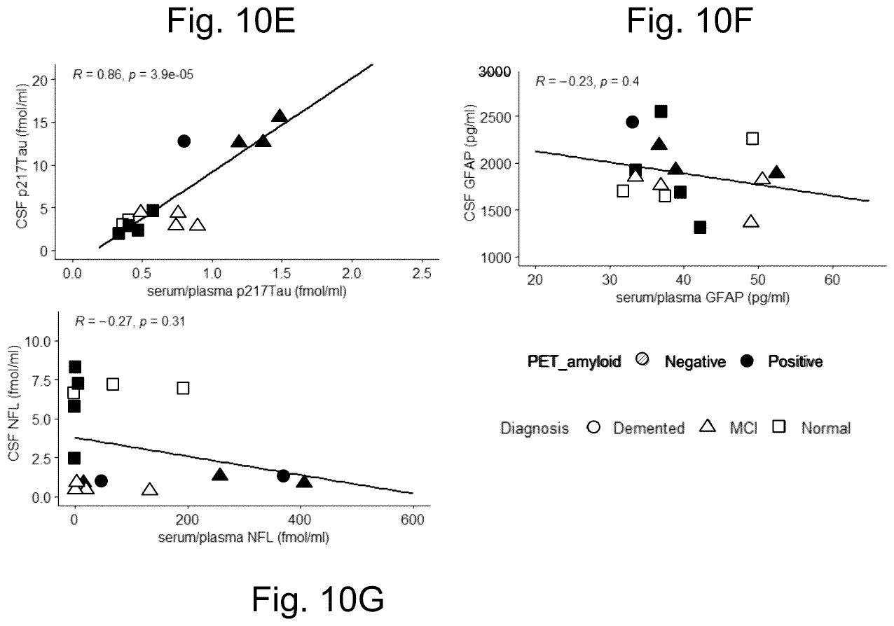

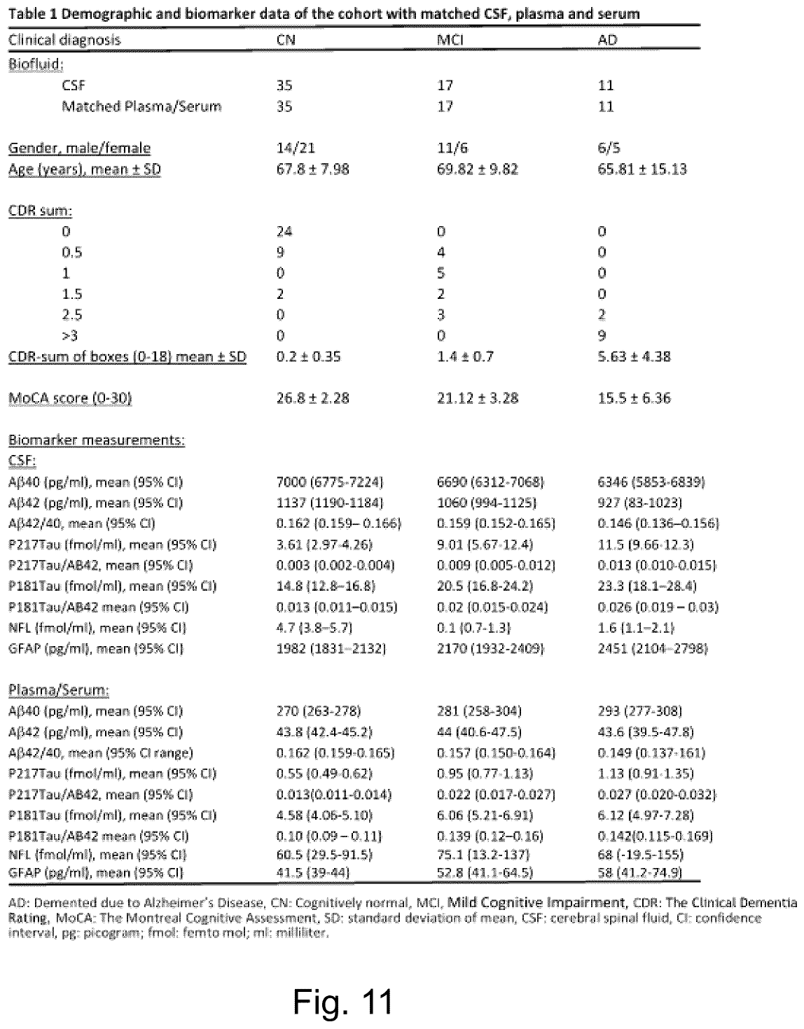

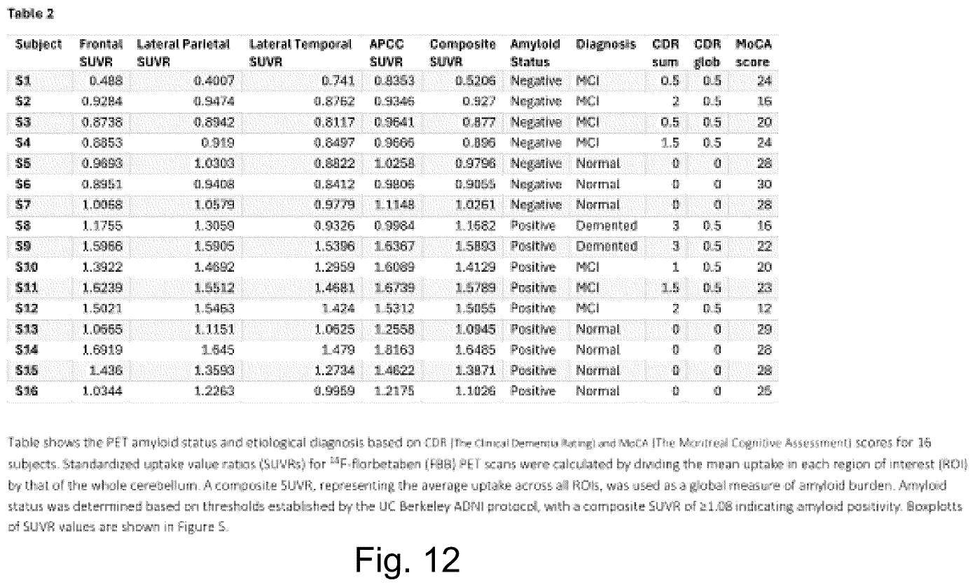

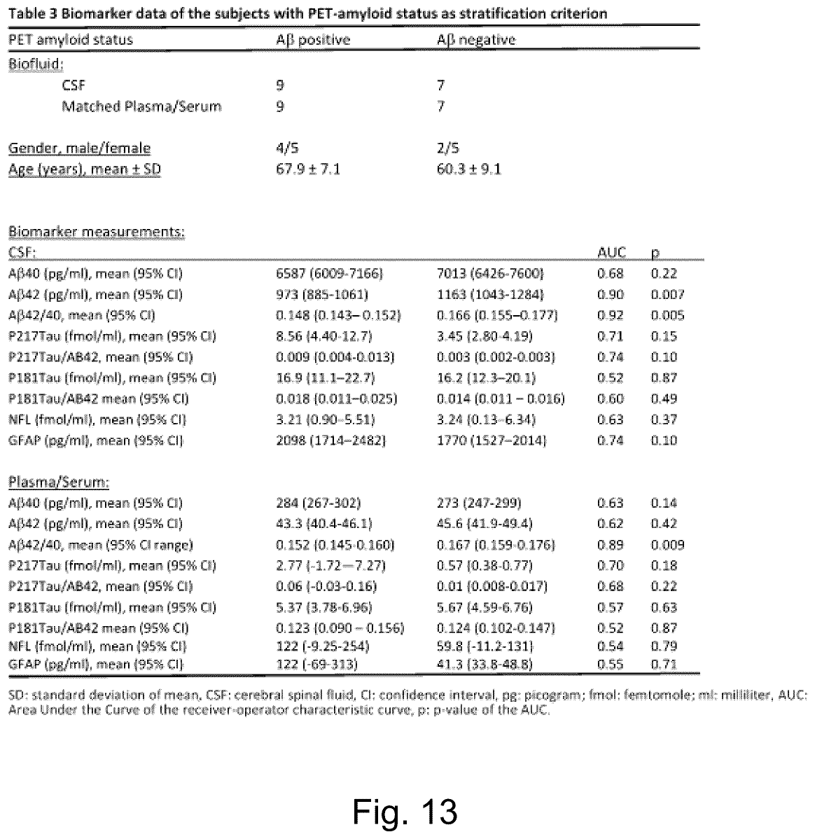

For 16 subjects from the cohort analyzed above,¹⁸F-florbetaben (FBB) brain positron tomography (PET) amyloid scans were available. We utilized the PET amyloid of this small subset of subjects (Table 2 and 3) to further validate the CSF and plasma/serum quantifications by 5ADCSI. The images were analyzed as described in the Supplementary Materials and Methods. The subjects were classified as PET amyloid negative (Aβ−) or positive (Aβ+) based on calculated standardized uptake value ratios (SUVRs) (Table 2, and FIG. 8). We utilized the brain amyloid status of the subjects (n=7 for Aβ negative and n=9 for Aβ positive) as the criteria for stratification of their blood biomarker analysis (Table 3 and FIG. 9). A comparison of each analyte measurement between the data presented in FIG. 6 (stratification based on clinical diagnosis) to the data in FIG. 9 indicates that the AUC of Aβ42/40 ratios is unchanged in the plasma/serum samples, and it increases from 0.8 (p=0.001) to 0.92 (p=0.005) in CSF samples. The AUC of other analytes have decreased when data are stratified based on PET amyloid status (FIG. 9). Importantly, however, the correlation of analyte values between CSF and plasma/serum for Aβ42 (r=0.5, p=0.047), Aβ42/40 (r=0.77, p=0.0005), p181Tau (r=0.63, p=0.009), and p217Tau (r=0.86, p=3.9e⁻⁵) are statistically significant (FIG. 10). Interestingly, GFAP (r=−0.23, p=0.4) and NfL (r=−0.27, p=0.31) show weak negative correlation (FIG. 10).

In summary, the validation experiments demonstrate that the 5ADCSI method quantifies key biomarkers (Aβ40, Aβ42, p181Tau, p217Tau, NfL, and GFAP) across matched CSF, plasma, and serum samples, distinguishing between CN, MCI, and AD cohorts. Biomarker levels and ratios, particularly Aβ42/40 and p217Tau, p217Tau/Aβ42, p181Tau, and p181/Aβ42, show significant agreement between CSF and plasma/serum samples, with high AUCs for differentiating clinical diagnoses. NfL exhibited notable variability, and GFAP showed weaker correlations.

### Discussion

The 5ADCSI multiplex system showed excellent specificity, sensitivity, and reliability for the quantification of six AD plasma biomarkers Aβ40, Aβ42, p181Tau, p217Tau, NfL, and GFAP in CSF and plasma and serum. To our knowledge there is no published report or commercial kit for the p217Tau phospho-antibody used here with xMAP technology. Thus, the 5ADCSI with the p217Tau is a new alternative to other assay systems available for p217Tau analysis. The 5ADCSI system either with p181Tau or p217Tau may provide an excellent and affordable diagnostic platform for Alzheimer's disease and related conditions.

The specificity of capture antibodies for 5ADCSI assay were confirmed at different ranges of analyte concentrations for specific antibody-target interactions. We used synthetic phosphorylated peptides for p181Tau and p217Tau to avoid concerns associated with variability due to incomplete phosphorylation of recombinant proteins in vitro. We also used a synthetic peptide as calibrator for NfL to improve quantification accuracy and to overcome variability caused by partial insolubility of recombinant NfL protein. Depletion with analyte-specific antibodies and phosphatase experiments for p181Tau and p217Tau further validated the reliability of 5ADCSI.

The 5ADCSI system's high sensitivity is evidenced by low LoQs and consistent detection and quantification of analytes at lower or near corresponding LoQs in plasma and serum (FIG. 5). It is important to note that LoQs of all 5ADCSI analytes are significantly below the levels of quantified biomarkers in plasma or serum (Table 1). Additionally, the inter- and intra-assay variability of <5% are in line with the precision benchmarks set by Ashton et al. (2021)²³, supporting the 5ADCSI's reproducibility and robustness.

Quantitative analyses of Aβ42/40 ratios, p217Tau, p217Tau/Ab42, p181Tau, p181Tau/Ab42, and GFAP of matched CSF and plasma or serum resulted in analyte values strongly differentiating among diagnostic groups with diagnostic accuracies ranging from AUCs=0.75 to 0.99 (FIG. 6). The data are in line with published reports^{3,24,10,25,26}. Very importantly, all biomarkers, except for NfL, correlated well between CSF and plasma or serum values calculated in this work (r=0.57-0.78) and align well with reported data of matched CSFs and plasma samples, where the performance of different assay platforms were compared^{3,24,10,27}.

It is well established that the Aβ42/40 ratios are lower in subjects with AD³. In this work, the Aβ42/40 ratios are 0.098 (CSF) and 0.083 (serum/plasma) fold lower in AD subjects compared to CN. These data are consistent with published reports^{3,26}. The diagnostic accuracies of Aβ42/40 ratios replicates the data in Janelidze et al. (2021) and Schindler et al. (2019) studies^{3,26}. We also observed similar trend for Aβ42/40 ratios when subjects are stratified based on PET-amyloid status (Table 3 and FIGS. 9 and 10).

Recent studies suggest that among other biofluid biomarkers, the p217Tau is the strongest diagnostic differentiator between subjects with MCI or AD from the CN individuals^{24,25,28}. In this work, in CSF the p217Tau mean values are 2.5-fold higher in MCI, and 3.2-fold higher in AD (AUC=0.91, p=0.0001) subjects. The p217Tau values in matched serum or plasma of MCI and AD are 1.72-fold and 2-fold higher than CN, respectively, with a diagnostic accuracy of AUC=0.95 (p=0.0001). The Pearson correlation between CSF and serum/plasma values for p217Tau are strong (R=0.75, p=2.5e⁻¹²). These data are consistent with other reports highlighting the p217Tau as a strong predictor of MCI and AD^{25,24,28}. Since we measured p217Tau and Aβ42 in the same well, we also calculated the p217Tau/Aβ42 ratios. The p217Tau/Aβ42 ratios improve by 20% and 35% in each corresponding group (FIG. 6 and Table 1) with significant diagnostic accuracies of 0.94 (serum/plasma) and 0.99 (CSF).

The p181Tau values in CSF are 1.38-, and 1.57-fold higher in MCI and AD compared to CN subjects, respectively, with AUCs of 0.81 (CSF), and 0.76 (serum/plasma), which are in line with data published by (Janelidze et al. (2023)²⁴.

When stratified based on PET amyloid status, the p217 Tau values of Aβ+ are 2.48-fold higher than Aβ− subjects in CSF (AUC=0.71, p=0.15), and 4.7-folds (AUC=0.7, p=0.18) higher in serum/plasma, in line with the data when subjects are classified based on clinical diagnosis. However, the fold changes of p181Tau values from Aβ+ and Aβ− (Table 3) groups were not consistent with those when subjects were classified by clinical diagnosis. This observation agrees with Montoliu-Gaya et al (2023) demonstrating that levels of p181Tau in plasma do not correlate with PET amyloid positivity, whereas there was a strong correlation for p217Tau, p205Tau, and p231Tau²⁹.

Our data of p217 and p181Tau biomarkers indicate higher diagnostic accuracy of p217Tau over p181Tau and aligns with data from Janelidze et al. (2023), showing better AUCs for p217Tau in predicting Alzheimer's pathology compared to p181Tau²⁴.

Our GFAP quantification show moderate diagnostic accuracy among CSFs and serum/plasmas across all subjects (CSF: AUC=0.78, p=0.005, serum/plasma: AUC=0.73, p=0.03), and Pearson correlation (r=0.59, p=8.2e⁻⁷), which are very well aligned with the Pereira et al (2021) who used HD-X (Quanterix®) for plasma GFAP and Elecsys assays (NeuroToolKit robust prototype, Roche Diagnostics) for CSF GFAP quantification in a larger cohort of Aβ+ and Aβ− individuals²⁷. They reported AUC=0.76 for plasma, and AUC=0.69 for CSF subjects³⁰.

The weaker results for NfL may indicate this protein has limited specificity in distinguishing amyloid pathology, despite its value in reflecting neuroinflammation and axonal injury^{31,32} The NfL may have limited value in diagnosing MCI and AD in this study due to its small sample size. However, its simultaneous measurement along with other AD biomarkers by 5ADCSI is valuable for study designs for other neurological diseases injuries.

The main objective of this work is to provide a first phase validation for the utility of 5ADCSI as an alternative to the existing assay platforms for quantification of AD biomarkers from CSF, serum and plasma. The clinical cohort used here is small for the biomarkers' data to have clinical relevancy. None the less, the diagnostic accuracies for Aβ42/Aβ40 ratio, p217Tau, p181Tau, and GFAP are statistically highly significant, and Pearson correlations between CSF and serum/plasma samples are strong and align well with published literature for these biomarkers^{25,24,3,27} Studies with much larger clinical cohort are needed to validate 5ADCSI as a potential clinical diagnostic method

In conclusion, the 5ADCSI assay platform is based on one of the most widely available immunological multiplex systems. The accessibility, affordability, and multiplexing ability coupled with high sensitivity and accuracy of 5ADCSI provide an excellent assay platform for affordable large-scale longitudinal research and clinical studies for accurate validation of blood-based biomarkers for early detection of AD dementia, and other neurological diseases.

### Supplementary Materials and Methods:

### Supplementary Figures and Table

### Clinical and Neuropsychological Assessment of all Participants

Participants were enrolled in the University of Southern California Alzheimer's Disease Research Center (USC ADRC) where they were evaluated and diagnosed based on history, physical examination, cognitive and functional assessment. Syndromal and etiological diagnoses were made per the National Alzheimer's Coordinating Center and ADRC protocol. Participants are first characterized by their syndrome as fulfilling criteria for dementia, core criteria for mild cognitive impairment, or as not cognitively impaired, using NIA-Alzheimer's Association draft research criteria^{13,14} Participants with dementia or MCI were then assessed for an etiologic diagnosis. Those for filling criteria for probable Alzheimer's disease dementia or MCI due to Alzheimer's disease were included. Participants without cognitive or functional impairment, who did not meet criteria for MCI or for dementia, had a global Clinical Dementia Rating scale score equal to zero, and neuropsychological test results within the normal range were considered as CN and included.

### CSF and Plasma/Serum Collection and Processing

CSF, plasma, and serum were collected the same day, in the morning at least 2 hours after last meal, with a difference of approximately 4 hours between them. The USC ADRC follows the ADRC/NACC procedures manual for sample collection and storage (https://naccdata.org/adrc-resources/best-practices). The lumbar puncture was performed using a standard 22-G needle, in lateral decubitus, between the L3 and L5 spaces. The CSF was drawn into 15-ml polypropylene tubes and centrifuged at room temperature at 2000 g for 10 min. Samples were aliquoted and frozen at −80° C.

Blood is collected into 10-ml EDTA tubes for plasma and red topped tubes for serum. Immediately after the blood is collected the EDTA tube for plasma is gently inverted 8 to 10 times in 180 degree turns. The samples are centrifuged at 2000 rpm for 20 minutes at 4° C., and aliquots are stored at −80° C. For serum, the blood is allowed to clot for 30 minutes and centrifuged at 4° C. for 10 minutes. The supernatant is collected, and aliquots frozen at −80° C.

### Image Analysis

Accelerated sagittal MPRAGE (3T), 18F-florbetaben (FBB), and 18F-flortaucipir (AV-1451) PET scans were sourced from the LONI-IDA repository, managed by the University of Southern California Laboratory of Neuroimaging (https://ida.loni.usc.edu/). PET scans collected within 6 months of the respective T1-weighted MRI were selected for further processing.

### T1 MRI Processing

T1-weighted images were processed using FreeSurfer version 7.1.1 (https://surfer.nmr.mgh.harvard.edu/). The resulting segmentations were quality checked and transformed to native space for further analysis.

### Amyloid PET Processing

18F-florbetaben (FBB) PET scans were processed according to the ADNI FBB protocol (https://adni.bitbucket.io/reference/docs/UCBERKELEYFBB/UCBerkeley_FBB_Methods_01.1 4.21.pdf). PET timeframes were motion-corrected, averaged, and smoothed using a 6 mm Gaussian kernel.

For region of interest (ROI) analysis, the Desikan-Killiany atlas was thresholded to extract voxels corresponding to the frontal, lateral parietal, lateral temporal, and anterior-posterior cingulate regions. The whole cerebellum was selected as the reference region and created by merging Freesurfer defined labels corresponding to bilateral cerebellar gray and white matter. To minimize partial volume effects (PVE), the resulting mask was eroded by one voxel. Each ROI and reference region was registered to PET space using the advanced normalization toolbox (ANTs) (Tustison et al., 2021) to facilitate uptake analysis.

Standardized uptake value ratios (SUVRs) for FBB were calculated by dividing the mean uptake in each ROI by that of the whole cerebellum. A composite SUVR, representing the average uptake across all ROIs, was used as a global measure of amyloid burden. Amyloid status was determined based on thresholds established by the UC Berkeley ADNI protocol, with a composite SUVR of ≥1.08 indicating amyloid positivity.

### Sequence of the Peptides Standard and Preparation

The sequences of peptide standards are listed in table 4 below. The HJ5.1-HA (referred to HJ-HA in main manuscript) is a hybrid peptide composed of the n-terminal HJ5.1 (amino acids 2-20 see table 4 below) portion (the sequence of tis portion was derived from chain A, Amyloid beta-peptide (Seq.ID: 1AMB_A), and HA (hemagglutinin) sequence, which corresponds to the amino acids 98-106 of the human influenza virus HA protein. The HJ5.1 portion is recognized by a HJ5.1 monoclonal antibody, and HA is recognized by HA antibody. The lyophilized white powder of the peptides were resuspended in the appropriate buffer solution according to specifications given in Table 5 and incubated on ice for 60 min. The concentration of peptides was determined using the Pierce quantitative colorimetric peptide assay kit (ThermoFisher Scientific, Cat #: 23275). Based on the concentration of each peptide, peptides were diluted to 10 ng/ml concentration using an appropriate dilution buffer and then 100 μl of 10 ng/ml each peptide was aliquoted in the microcentrifuge tubes and kept in the −20 C for further use. For GFAP we used Gst-GFAP (Proteintech, Cat #16825-1-AP).

TABLE 4
Peptide sequences

| Peptide | | | Cat No./ |
|---|---|---|---|
| Name | N-C Sequence | Purity | Company |
| HJ5.1HA | DAGYEVHHQKLVFFAEDVGSEYPYDVPDY | >96% | A-5749-1/ |
| peptide | AAKLE (SEQ ID NO: 2) | | Thermo- |
| | | | Fisher |
| Beta- | DAEFRHDSGYEVHHQKLVFFAEDVGSNKG | >96% | AS-24235/ |
| Amyloid (1- | AIIGLMVGGVV (SEQ ID NO: 5) | | Anaspec |
| 40) peptide | | | |
| Beta- | DAEFRHDSGYEVHHQKLVFFAEDVGSNKG | >95% | AS-20276/ |
| Amyloid (1- | AIIGLMVGGVVIA (SEQ ID NO: 6) | | Anaspec |
| 42) peptide | | | |
| Tau12T181 | AEPRQEFEVMEDHAGTYGLGDTPPAPKTPP | >98% | A6307-1/ |
| | SSGEPPK (SEQ ID NO: 7) | | Thermo- |
| | | | Fisher |
| Tau12pT181 | AEPRQEFEVMEDHAGTYGLGDTPPAPKT[(p | >98% | A6307-2/ |
| | T)]PPSSGEPPK (SEQ ID NO: 1) | | Thermo- |
| | | | Fisher |
| Tau12T217 | AEPRQEFEVMEDHAGTYGLGDRSRTPSLPT | >98% | A6307- |
| | PPTREPK (SEQ ID NO: 8) | | 3/Thermo- |
| | | | Fisher |
| Tau12pT217 | AEPRQEFEVMEDHAGTYGLGDRSRTPSLP(p | >98% | A6307- |
| | T)PPTREPK (SEQ ID NO: 4) | | 4/Thermo- |
| | | | Fisher |
| NFL_UD1_2 | RLLKAKTLEIEACRGMNEALEKQLQELEDK | >95% | AAPPTEC |
| | QNADISAMQSTINKLENELRTTK (SEQ ID | | |
| | NO: 4) | | |

TABLE 5
Resuspension and dilution buffers for peptides

| Peptides | Buffer solution for | Buffer solution for |
|---|---|---|
| Name | resuspension | dilution |
| HJ5.1HA | 1X PBS, 200 mM | 0.1 mg/ml BSA, 1X PBS, |
| peptide | EPPS, pH 7.4 | 200 mM EPPS, pH 7.4 |
| Beta- | 0.1% NH4OH:ACN | 0.1 mg/ml BSA, 0.1% |
| Amyloid (1- | (4:1) | NH4OH:ACN (4:1) |
| 40) peptide | | |
| Beta- | 0.1% NH4OH:ACN | 0.1 mg/ml BSA, 0.1% |
| Amyloid (1- | (4:1) | NH4OH:ACN (4:1) |
| 42) peptide | | |
| Tau12T181 | 100 mM Sodium | 0.1 mg/ml BSA, 100 mM |
| | bicarbonate:ACN (4:1) | Sodium bicarbonate:ACN (4:1) |
| Tau12pT181 | 100 mM Sodium | 0.1 mg/ml BSA, 100 mM |
| | bicarbonate:ACN (4:1) | Sodium bicarbonate:ACN (4:1) |
| NFL_UD1_{—} | 100 mM Sodium | 0.1 mg/ml BSA, 100 mM |
| 2 | bicarbonate:ACN (4:1) | Sodium bicarbonate:ACN (4:1) |

### Peptides and Proteins Dilution for Standard Curve

Peptides dilution and standards were prepared by diluting peptides stock solution in the standard dilution buffer: 20 mg/ml BSA, 1X PBS, 200 mM EPPS [4-(2-Hydroxyethyl)-1-piperazinepropanesulfonic acid, 4-(2-Hydroxyethyl)piperazine-1-propanesulfonic acid, N-(2-Hydroxyethyl)piperazine-N′-(3-propanesulfonic acid)], 250 mM guanidine hydrochloride (GuHCl), 0.225% CHAPS (3-[(3-Cholamidopropyl) dimethylammonio]-1-propanesulfonate hydrate), and 2 mM EDTA (Ethylenedinitrilo)tetraacetic acid), 0.05% sodium Azide, pH 7.4. To prepare standards, 1000pg/ml peptides mixture was prepared by spiking 1000 μg/ml of each peptide in dilution buffer and then serially diluting in 1:1 (v/v) ratio with the same dilution buffer.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Pais M V, Forlenza O V, Diniz B S. Plasma Biomarkers of Alzheimer's Disease: A Review of Available Assays, Recent Developments, and Implications for Clinical Practice. J Alzheimers Dis Rep. 2023; 7(1):355-380. doi:10.3233/ADR-230029
2. Ovod V, Ramsey K N, Mawuenyega K G, et al. Amyloid β concentrations and stable isotope labeling kinetics of human plasma specific to central nervous system amyloidosis. Alzheimers Dement. August 2017; 13(8):841-849. doi:10.1016/j.jalz.2017.06.2266
3. Janelidze S, Teunissen C E, Zetterberg H, et al. Head-to-Head Comparison of 8 Plasma Amyloid-β 42/40 Assays in Alzheimer Disease. JAMA Neurol. Sep. 20, 2021; doi:10.1001/jamaneurol.2021.3180
4. Suarez-Calvet M, Karikari T K, Ashton N J, et al. Novel tau biomarkers phosphorylated at T181, T217 or T231 rise in the initial stages of the preclinical Alzheimer's continuum when only subtle changes in A beta pathology are detected. EMBO MOLECULAR MEDICINE. Dec. 7, 2020; 12(12)doi:10.15252/emmm.202012921
5. Karikari T K, Pascoal T A, Ashton N J, et al. Blood phosphorylated tau 181 as a biomarker for Alzheimer's disease: a diagnostic performance and prediction modelling data from four cohorts. LANCET NEUROLOGY. May 2020; 19(5):422-433.
6. Palmqvist S, Tideman P, Cullen N, et al. Prediction of future Alzheimer's disease dementia using plasma phospho-tau combined with other accessible measures. Nat Med. June 2021; 27(6):1034-1042. doi:10.1038/s41591-021-01348-z
7. Pichet Binette A, Palmqvist S, Bali D, et al. Combining plasma phospho-tau and accessible measures to evaluate progression to Alzheimer's dementia in mild cognitive impairment patients. Alzheimers Res Ther. Mar. 29, 2022; 14(1):46. doi:10.1186/s13195-022-00990-0
8. Chatterjee P, Pedrini S, Doecke J D, et al. Plasma Aβ42/40 ratio, p-tau181, GFAP, and NfL across the Alzheimer's disease continuum: A cross-sectional and longitudinal study in the AIBL cohort. Alzheimers Dement. April 2023; 19(4):1117-1134. doi:10.1002/alz.12724
9. Zhang H, Liu J, Zhang N, Jeromin A, Lin Z J. Validation of an Ultra-Sensitive Method for Quantitation of Phospho-Tau 217 (pTau217) in Human Plasma, Serum, and CSF using the ALZpath pTau217 Assay on the Quanterix HD-X Platform. J Prev Alzheimers Dis. 2024; 11(5):1206-1211. doi:10.14283/jpad.2024.155
10. Janelidze S, Barthélemy NR, Salvadó G, et al. Plasma Phosphorylated Tau 217 and Aβ42/40 to Predict Early Brain Aβ Accumulation in People Without Cognitive Impairment. JAMA Neurol. Sep. 1, 2024; 81(9):947-957. doi:10.1001/jamaneurol.2024.2619
11. Noda K, Lim Y, Goto R, Sengoku S, Kodama K. Cost-effectiveness comparison between blood biomarkers and conventional tests in Alzheimer's disease diagnosis. Drug Discov Today. Feb. 2, 2024:103911. doi:10.1016/j.drudis.2024.103911
12. Kang J H, Vanderstichele H, Trojanowski J Q, Shaw L M. Simultaneous analysis of cerebrospinal fluid biomarkers using microsphere-based xMAP multiplex technology for early detection of Alzheimer's disease. Methods. April 2012; 56(4):484-93. doi:10.1016/j.ymeth.2012.03.023
13. Park J C, Han S H, Cho H J, et al. Chemically treated plasma Aβ is a potential blood-based biomarker for screening cerebral amyloid deposition. Alzheimers Res Ther. Mar. 22, 2017; 9(1):20. doi:10.1186/s13195-017-0248-8
14. Lövheim H, Elgh F, Johansson A, et al. Plasma concentrations of free amyloid p cannot predict the development of Alzheimer's disease. Alzheimers Dement. July 2017; 13(7):778-782. doi:10.1016/j.jalz.2016.12.004
15. Bachmann S, Bell M, Klimek J, Zempel H. Differential Effects of the Six Human TAU Isoforms: Somatic Retention of 2N-TAU and Increased Microtubule Number Induced by 4R-TAU. Front Neurosci. 2021; 15:643115. doi:10.3389/fnins.2021.643115
16. Shaw G, Madorsky I, Li Y, et al. Uman-type neurofilament light antibodies are effective reagents for the imaging of neurodegeneration. Brain Commun. 2023; 5(2):fcad067. doi:10.1093/braincomms/fcad067
17. Cipollari E, Szapary H J, Picataggi A, et al. Correlates and Predictors of Cerebrospinal Fluid Cholesterol Efflux Capacity from Neural Cells, a Family of Biomarkers for Cholesterol Epidemiology in Alzheimer's Disease. JOURNAL OF ALZHEIMERS DISEASE. 2020; 74(2):563-578. doi:10.3233/JAD-191246
18. Fazeli B, Huss A, Gómez de San José N, Otto M, Tumani H, Halbgebauer S. Development of an ultrasensitive microfluidic assay for the analysis of Glial fibrillary acidic protein (GFAP) in blood. Front Mol Biosci. 2023; 10:1175230. doi:10.3389/fmolb.2023.1175230
19. Armbruster D A, Pry T. Limit of blank, limit of detection and limit of quantitation. Clin Biochem Rev. August 2008; 29 Suppl 1(Suppl 1):549-52.
20. Iino T, Watanabe S, Yamashita K, et al. Quantification of Amyloid-β in Plasma by Simple and Highly Sensitive Immunoaffinity Enrichment and LC-MS/MS Assay. J Appl Lab Med. Jul. 7, 2021; 6(4):834-845. doi:10.1093/jalm/jfaa225
21. West T, Kirmess K M, Meyer M R, et al. A blood-based diagnostic test incorporating plasma A beta 42/40 ratio, ApoE proteotype, and age accurately identifies brain amyloid status: findings from a multi cohort validity analysis. MOLECULAR NEURODEGENERATION. May 1, 2021; 16(1)doi:10.1186/s13024-021-00451-6
22. Planche V, Bouteloup V, Pellegrin I, et al. Validity and Performance of Blood Biomarkers for Alzheimer Disease to Predict Dementia Risk in a Large Clinic-Based Cohort. Neurology. Jan. 31, 2023; 100(5):e473-e484. doi:10.1212/WNL.0000000000201479
23. Ashton N J, Leuzy A, Karikari T K, et al. The validation status of blood biomarkers of amyloid and phospho-tau assessed with the 5-phase development framework for AD biomarkers. EUROPEAN JOURNAL OF NUCLEAR MEDICINE AND MOLECULAR IMAGING. Jul. 2021; 48(7):2140-2156. doi:10.1007/s00259-021-05253-y
24. Janelidze S, Bali D, Ashton N J, et al. Head-to-head comparison of 10 plasma phospho-tau assays in prodromal Alzheimer's disease. Brain. Apr. 19, 2023; 146(4):1592-1601. doi:10.1093/brain/awac333
25. Barthélemy NR, Salvadó G, Schindler S E, et al. Highly accurate blood test for Alzheimer's disease is similar or superior to clinical cerebrospinal fluid tests. Nat Med. April 2024; 30(4):1085-1095. doi:10.1038/s41591-024-02869-z
26. Schindler S E, Bollinger J G, Ovod V, et al. High-precision plasma β-amyloid 42/40 predicts current and future brain amyloidosis. Neurology. 10 22 2019; 93(17):e1647-e1659. doi:10.1212/WNL.0000000000008081
27. Pereira J B, Janelidze S, Smith R, et al. Plasma GFAP is an early marker of amyloid-β but not tau pathology in Alzheimer's disease. Brain. Dec. 16, 2021; 144(11):3505-3516. doi:10.1093/brain/awab223
28. Mendes A J, Ribaldi F, Lathuiliere A, et al. Head-to-head study of diagnostic accuracy of plasma and cerebrospinal fluid p-tau217 versus p-tau181 and p-tau231 in a memory clinic cohort. J Neurol. April 2024; 271(4):2053-2066. doi:10.1007/s00415-023-12148-5
29. Montoliu-Gaya L, Benedet A L, Tissot C, et al. Mass spectrometric simultaneous quantification of tau species in plasma shows differential associations with amyloid and tau pathologies. Nat Aging. June 2023; 3(6):661-669. doi:10.1038/s43587-023-00405-1
30. Cheng Y, Pereira M, Raukar N, et al. Potential biomarkers to detect traumatic brain injury by the profiling of salivary extracellular vesicles. JOURNAL OF CELLULAR PHYSIOLOGY. August 2019; 234(8):14377-14388. doi:10.1002/jcp.28139
31. Mattsson-Carlgren N, Grinberg L T, Boxer A, et al. Cerebrospinal Fluid Biomarkers in Autopsy-Confirmed Alzheimer Disease and Frontotemporal Lobar Degeneration. Neurology. Mar. 15, 2022; 98(11):e1137-e1150. doi:10.1212/WNL.0000000000200040
32. Pichet Binette A, Janelidze S, Cullen N, et al. Confounding factors of Alzheimer's disease plasma biomarkers and their impact on clinical performance. Alzheimers Dement. April 2023; 19(4):1403-1414. doi:10.1002/alz.12787
33. R Core Team (2024). _R: A Language and Environment for Statistical Computing_. R Foundation for Statistical Computing, Vienna, Austria, <https://www.R-project.org/

What is claimed is:

1. A bioassay system for multiplexed detection and quantification of multiple analytes in a biological sample, the bioassay system comprising:
a plurality of sets of color-coded microspheres, the plurality of sets of color-coded microspheres including a first set of microspheres distinguishable by a first unique color code generated by internal dyes, each microsphere in the first set being coated with a specific capture agent that binds to Aβ40, a second set of microspheres distinguishable by a second unique color code generated by internal dyes, each microsphere in the second set being coated with a specific capture agent that binds to Aβ42, a third set of microspheres distinguishable by a third unique color code generated by internal dyes, each microsphere in the third set being coated with a specific capture agent that binds to p181Tau or p217Tau, a fourth set of microspheres distinguishable by a fourth unique color code generated by internal dyes, each microsphere in the fourth set being coated with a specific capture agent that binds to GFAP, and a fifth set of microspheres distinguishable by a fifth unique color code generated by internal dyes, each microsphere in the fifth set being coated with a specific capture agent that binds to Neurofilament Light Chain (NFL);
a fluidic system configured to mix the biological sample with the plurality of sets of color-coded microspheres to allow for specific binding between analytes and their corresponding capture agent among other analytes and their respective capture agents, and to introduce reporter molecules that bind to captured analytes, the reporter molecules configured to emit a detectable reporter fluorescence upon excitation; and
a detection system comprising a flow cell and capable of exciting and reading fluorescence of each internal dye and the detectable reporter fluorescence indicative of analyte binding,
wherein the detection system is configured to identify each of the sets of microspheres by its associated unique color code and quantify bound analytes based on the detectable reporter fluorescence, alongside quantification of other analytes.

2. The bioassay system of claim 1 further comprising a first set of control microspheres attached to mouse polyclonal IgG to correct for a background of individual specimens.

3. The bioassay system of claim 1, wherein a specificity test for p217Tau applies a calibrator having a sequence AEPRQEFEVMEDHAGTYGLGDRSRTPSLP(pT)PPTREPK (SEQ ID NO: 4).

4. The bioassay system of claim 1, wherein a calibrator for p181Tau specificity testing comprises the sequence N-AEPRQEFEVMEDHAGTYGLGDTPPAPKpTPPSSGEPPK (SEQ ID NO: 1).

5. The bioassay system of claim 1 further comprising a second set of control microspheres configured to capture a synthetic peptide to normalize for well-to-well variations.

6. The bioassay system of claim 5, wherein the synthetic peptide includes a peptide having a sequence DAGYEVHHQKLVFFAEDVGSEYPYDVPDYAAKLE (SEQ ID NO: 2) or a peptide at least 90% identical to SEQ ID NO: 2 without any substitution in any epitopes therein.

7. The bioassay system of claim 5, wherein the synthetic peptide includes a peptide having a sequence DYKDHDIDYKDDDDKGGGEYPYDVPDYAAKLE (SEQ ID NO: 3) or a peptide at least 90% identical to SEQ ID NO: 3 without any substitution in any epitopes therein.

8. The bioassay system of claim 1, wherein the specific capture agent is an antibody.

9. The bioassay system of claim 1, wherein the reporter molecules include a detection antibody that is biotinylated.

10. The bioassay system of claim 1, wherein the reporter molecules include a fluorescent moiety.

11. The bioassay system of claim 1, wherein the specific capture agents for Aβ40, Aβ42, either pTau181 or p217Tau, GFAP, and NFL are antibodies that specifically bind to their respective analytes, allowing for selective detection and quantification of these analytes in the presence of other analytes within the biological sample.

12. The bioassay system of claim 1, wherein the biological sample is a cerebrospinal fluid sample or a plasma sample.

13. The bioassay system of claim 1, wherein the detection system includes a first laser for exciting internal dyes and a second laser for exciting reporter fluorescence.

14. The bioassay system of claim 1, further comprising a data analysis system configured to normalize signals using control microspheres and to calculate ratios between different analytes, wherein the ratios include at least one of: Aβ42/Aβ40, p217Tau/Aβ42, and p181Tau/Aβ42.

15. The bioassay system of claim 1, wherein the system has a limit of quantification of less than 5 μg/ml for Aβ40, Aβ42, and GFAP, and less than 0.3 fmol/ml for p181Tau, p217Tau, and NFL.

16. The bioassay system of claim 1, wherein the reporter molecules include Streptavidin R-phycoerythrin conjugate (SAPE).

17. A bioassay system for multiplexed detection and quantification of multiple analytes in a biological sample, the bioassay system comprising:
a plurality of sets of color-coded microspheres, the plurality of sets of color-coded microspheres including a first set of microspheres distinguishable by a first unique color code generated by internal dyes, each microsphere in the first set being coated with a specific capture agent that binds to Aβ40, a second set of microspheres distinguishable by a second unique color code generated by internal dyes, each microsphere in the second set being coated with a specific capture agent that binds to Aβ42, a third set of microspheres distinguishable by a third unique color code generated by internal dyes, each microsphere in the third set being coated with a specific capture agent that binds to p217Tau, a fourth set of microspheres distinguishable by a fourth unique color code generated by internal dyes, each microsphere in the fourth set being coated with a specific capture agent that binds to GFAP, and a fifth set of microspheres distinguishable by a fifth unique color code generated by internal dyes, each microsphere in the fifth set being coated with a specific capture agent that binds to Neurofilament Light Chain (NFL);
a fluidic system configured to mix the biological sample with the plurality of sets of color-coded microspheres to allow for specific binding between analytes and their corresponding capture agent among other analytes and their respective capture agents, and to introduce reporter molecules that bind to captured analytes, the reporter molecules configured to emit a detectable reporter fluorescence upon excitation; and
a detection system comprising a flow cell and capable of exciting and reading fluorescence of each internal dye and the detectable reporter fluorescence indicative of analyte binding,
wherein the detection system is configured to identify each of the sets of microspheres by its associated unique color code and quantify bound analytes based on the detectable reporter fluorescence, alongside quantification of other analytes.

18. The bioassay system of claim 17, further comprising a first set of control microspheres attached to mouse polyclonal IgG to correct for background signal and a second set of control microspheres configured to capture a synthetic peptide to normalize for well-to-well variations.

19. The bioassay system of claim 17, further comprising a data analysis system configured to calculate a p217Tau/Aβ42 ratio from quantified analytes, wherein the p217Tau/Aβ42 ratio provides enhanced diagnostic accuracy for distinguishing Alzheimer's disease from cognitively normal individuals.

20. A method for multiplexed detection and quantification of multiple analytes in a biological sample, the method comprising:
mixing the biological sample with a plurality of sets of color-coded microspheres, the plurality of sets of color-coded microspheres including a first set of microspheres distinguishable by a first unique color code generated by internal dyes, each microsphere in the first set being coated with a specific capture agent that binds to Aβ40, a second set of microspheres distinguishable by a second unique color code generated by internal dyes, each microsphere in the second set being coated with a specific capture agent that binds to Aβ42, a third set of microspheres distinguishable by a third unique color code generated by internal dyes, each microsphere in the third set being coated with a specific capture agent that binds to p181Tau or p217Tau, a fourth set of microspheres distinguishable by a fourth unique color code generated by internal dyes, each microsphere in the fourth set being coated with a specific capture agent that binds to GFAP, and a fifth set of microspheres distinguishable by a fifth unique color code generated by internal dyes, each microsphere in the fifth set being coated with a specific capture agent that binds to Neurofilament Light Chain (NFL);
allowing specific binding between analytes in the biological sample and their corresponding capture agents on the microspheres;
introducing reporter molecules that bind to captured analytes, the reporter molecules configured to emit a detectable reporter fluorescence upon excitation;
passing the microspheres through a flow cell;
exciting and reading fluorescence of each internal dye and the detectable reporter fluorescence indicative of analyte binding; and
identifying each of the sets of microspheres by its associated unique color code and quantifying bound analytes based on the detectable reporter fluorescence.

21. The method of claim 20, further comprising adding a first set of control microspheres attached to mouse polyclonal IgG to correct for a background of individual specimens and a second set of control microspheres configured to capture a synthetic peptide to normalize for well-to-well variations.

22. The method of claim 20, further comprising calculating at least one ratio selected from the group consisting of: Aβ42/Aβ40 ratio, p217Tau/Aβ42 ratio, and p181Tau/Aβ42 ratio, wherein the ratios provide enhanced diagnostic accuracy for distinguishing between cognitively normal individuals, individuals with mild cognitive impairment, and individuals with Alzheimer's disease.
